(19) United States
(12) Patent Application Publication
(10) Pub. No.: US 2026/0137947 A1
(43) Pub. Date: May 21, 2026

(54) METHOD AND APPARATUS FOR MONITORING CONDUCTION SYSTEM PACING

(71) Applicant: Medtronic, Inc., Minneapolis, MN (US)

(72) Inventors: Keara BERLIN, St. Paul, MN (US); Jian CAO, San Jose, CA (US); Wade M. DEMMER, Coon Rapids, MN (US); Elizabeth Ann MATTSON, Lauderdale, MN (US)

(21) Appl. No.: 19/122,235

(22) PCT Filed: Oct. 5, 2023
(86) PCT No.: PCT/IB2023/059987

§ 371 Date: Apr. 17, 2025

Related U.S. Application Data
(60) Provisional application No. 63/381,268, filed on Oct. 27, 2022.

(51) Int. Cl. A61N 1/37; A61B 5/366; A61N 1/372

(52) U.S. Cl. CPC A61N 1/371; A61B 5/366; A61N 1/37247

(57) ABSTRACT

A medical device system is configured to obtain a cardiac electrical signal episode sensed during a conduction system capture management test and determine capture test data from the cardiac electrical signal episode. The medical device system may be configured to detect an alert condition of the conduction system capture management test indicating a change in the capture test data compared to a previous conduction system capture management test and generate an output in response to detecting the alert condition. The generated output may be stored in memory of the medical device system. The medical device system may display data in a user interface based on the generated output.

### TECHNICAL FIELD

This application claims the benefit of U.S. Provisional Patent Application Ser. No. 63/381,268, filed Oct. 27, 2022, the entire content of which is incorporated herein by reference.

This disclosure relates to a medical device system and method for monitoring conduction system pacing of a patient's heart.

### BACKGROUND

During normal sinus rhythm (NSR), the heartbeat is regulated by electrical signals produced by the sino-atrial (SA) node located in the right atrial wall. Each depolarization signal produced by the SA node spreads across the atria, causing the depolarization and contraction of the atria, and arrives at the atrioventricular (AV) node. The AV node responds by propagating a depolarization signal through the bundle of His (or “His bundle”) of the atrioventricular septum and thereafter to the bundle branches and the Purkinje muscle fibers of the right and left ventricles, sometimes referred to as the “His-Purkinje conduction system.”

Patients with a conduction system abnormality, e.g., poor AV node conduction, poor SA node function, or other conduction abnormalities, may receive a pacemaker to restore a more normal heart rhythm and heart chamber synchrony. Ventricular pacing may be performed to maintain the ventricular rate in a patient having atrioventricular conduction abnormalities. A single chamber ventricular pacemaker may be coupled to a transvenous ventricular lead carrying electrodes placed in the right ventricle (RV), e.g., in the right ventricular apex. The pacemaker itself is generally implanted in a subcutaneous pocket with the transvenous ventricular lead tunneled to the subcutaneous pocket. Intracardiac pacemakers have been introduced or proposed for implantation entirely within a patient's heart, eliminating the need for transvenous leads. An intracardiac pacemaker may provide sensing and pacing from within a chamber of the patient's heart, e.g., from within the right ventricle in a patient having AV conduction block.

Dual chamber pacemakers are available which include a transvenous atrial lead carrying electrodes which are placed in the right atrium and a transvenous ventricular lead carrying electrodes that are placed in the right ventricle via the right atrium. A dual chamber pacemaker senses atrial electrical signals and ventricular electrical signals and can provide both atrial pacing and ventricular pacing as needed to promote a normal atrial and ventricular rhythm and promote AV synchrony when SA and/or AV node or other conduction abnormalities are present.

Ventricular pacing via electrodes at or near the right ventricular apex has been found to be associated with increased risk of atrial fibrillation and heart failure. Alternative pacing sites have been investigated or proposed, such as pacing of the His bundle or left bundle branch. Ventricular pacing along the His-Purkinje conduction system has been proposed to provide a more physiologic form of ventricular pacing because pacing-evoked depolarizations can be conducted along the heart's natural conduction system. Pacing the ventricles via the His bundle or left bundle branch, for example, allows recruitment along the heart's natural conduction system, including the bundle branches and the Purkinje fibers, and is hypothesized to promote more physiologically normal cardiac activation than other pacing sites, such as at the ventricular apex.

### SUMMARY

The techniques of this disclosure generally relate to a medical device system for monitoring pacing of the His-Purkinje conduction system, also referred to herein as the “conduction system,” of a patient's heart. Conduction system pacing (CSP) may be delivered for pacing the ventricles by placing at least one electrode along or in the vicinity of the His-Purkinje conduction system, which may be along the His bundle or along or in the area of one or both of the left bundle branch (LBB) and/or right bundle branch (RBB). In various examples, a medical device system operating according to methods disclosed herein obtains a cardiac electrical signal episode sensed during a conduction system capture management (CM) test. The CM test may be performed by an implanted medical device coupled to and/or carrying electrodes positioned for delivering ventricular pacing via the conduction system. During the CM test, CSP pulse output, e.g., the pacing pulse amplitude and/or the pacing pulse width, may be varied. One or more cardiac electrical signals, which may be cardiac electrogram (EGM) signals sensed using one or more electrodes implanted in or on the heart, may be recorded as a cardiac electrical signal episode sensed during the CM test by the implanted medical device and transmitted to an external device, e.g., a computing device, for processing by medical device system processing circuitry.

The processing circuitry may be configured to analyze the cardiac electrical signal episode to determine capture test data. Various capture test data may include a number of different QRS waveform morphologies present in the cardiac electrical signal episode, a capture type classification of one or more of the QRS waveform morphologies, a capture threshold associated with one or more of the QRS waveform morphologies and/or capture type classifications identified in the cardiac electrical signal episode. The processing circuitry may identify one or more changes in the capture test data compared to the capture test data determined from a previous CM test. The processing circuitry may detect an alert condition corresponding to a change in the capture test data compared to the previous capture test data. The medical device system may generate an output based on the capture test data, which may include generating data for display in a user interface. The processing circuitry may receive user input, e.g., truth input, via the user interface to label QRS waveform morphologies according to morphology types and/or capture types. The processing circuitry may use the user input for identifying changes in the capture test data compared to a CM test and for detecting alert condition(s).

Further disclosed herein is the subject matter of the following examples:

Example 1. A medical device system including processing circuitry configured to receive a cardiac electrical signal episode sensed during a conduction system capture management test and determine capture test data from the cardiac electrical signal episode. The processing circuitry may compare the capture test data to previous capture test data determined from a previous conduction system capture management test generate an output based on the comparing of the capture test data to the previous capture test data; and a memory configured to store the output.

Example 2. The medical device system of example 1 wherein the processing circuitry is further configured to detect an alert condition corresponding to a change in the capture test data compared to the previous capture test data. The medical device system of example 1 may further include a display unit in communication with the processing circuitry. The display unit may receive the output generated by the processing circuitry to display data in a user interface based on the generated output and the alert condition.

Example 3. The medical device system of any of examples 1-2 wherein the processing circuitry is configured to determine the capture test data by identifying a number of different QRS waveform morphologies in the cardiac electrical signal episode.

Example 4. The medical device system of example 3 wherein the processing circuitry is further configured to detect an alert condition by determining that the number of different QRS waveform morphologies in the cardiac electrical signal episode is different than a previous number of QRS waveform morphologies identified in a previous cardiac electrical signal episode sensed during the previous conduction system capture management test.

Example 5. The medical device system of any of examples 3-4 wherein the processing circuitry is further configured to determine the capture test data by determining a capture threshold for one or more of the different QRS morphologies identified in the cardiac electrical signal episode.

Example 6. The medical device system of example 5 wherein the processing circuitry is further configured to detect an alert condition by determining that a capture threshold determined for at least one of the QRS morphologies identified in the cardiac electrical signal episode is different than a previously determined capture threshold determined for the respective QRS morphology identified in the previous cardiac electrical signal episode sensed during the previous capture management test.

Example 7. The medical device system of any of examples 1-6 wherein the processing circuitry is further configured to determine the capture test data by classifying at least one QRS waveform in the cardiac electrical signal episode according to a capture type.

Example 8. The medical device system of any of examples 1-7 wherein the processing circuitry is further configured to determine the capture test data by classifying a plurality of QRS waveforms in the cardiac electrical signal episode according to a plurality of capture types.

Example 9. The medical device system of example 8 wherein the processing circuitry is further configured to determine a capture threshold of one or more of the classified capture types of the plurality of capture types of the cardiac electrical signal episode.

Example 10. The medical device system of example 9, wherein the processing circuitry is further configured to detect the alert condition by determining a change in the capture threshold determined for at least one of the classified capture types compared to a previous capture threshold determined for the at least one of the classified capture types in the previous conduction system capture management test.

Example 11. The medical device of any of examples 8-10 wherein the processing circuitry is further configured to determine an alert condition by determining that a classified capture type of the plurality of capture types occurs at a pacing pulse output of a plurality of pacing pulse outputs of the conduction system capture management test and that the classified capture type of the plurality of capture types does not occur at the pacing pulse output of the plurality of pacing pulse outputs of the previous conduction system capture management test.

Example 12. The medical device system of any of examples 1-11 wherein the processing circuitry is further configured to receive, via the user interface, a user input labeling at least one QRS waveform of the cardiac electrical signal episode according to at least one of a morphology type or a capture type.

Example 13. The medical device system of example 12 wherein the processing circuitry is further configured to detect an alert condition based on the capture test data determined from the cardiac electrical signal episode and the user input.

Example 14. The medical device system of any of examples 12-13 wherein the processing circuitry is further configured to adjust the determination of capture test data from the cardiac electrical signal episode in response to receiving the user input.

Example 15. The medical device system of example 14 wherein the processing circuitry is further configured to adjust the determination of capture test data by adjusting a criteria applied to a QRS morphology of the cardiac electrical signal episode for detecting a morphology change.

Example 16. The medical device system of any of examples 14-15 wherein the processing circuitry is further configured to adjust the determination of capture test data by adjusting a criteria applied to a QRS morphology of the cardiac electrical signal episode for detecting a conduction system capture type.

Example 17. The medical device system of any of examples 1-16 wherein the processing circuitry is further configured to determine the capture test data by classifying each of a plurality of post-pace waveforms of the cardiac electrical signal episode according to a capture type selected as being one or more of: selective conduction system capture, non-selective conduction system capture, ventricular myocardial only capture without capture of the conduction system, left bundle branch capture, partial left bundle branch capture, right bundle branch capture, partial bundle branch capture, complete His bundle capture, partial His bundle capture or loss of capture.

Example 18. The medical device system of any of examples 1-17 further comprising a communication circuit configured to receive the cardiac electrical signal episode transmitted from an implantable medical device, the cardiac electrical signal episode including at least one cardiac electrogram signal.

Example 19. A method including receiving by processing circuitry of a medical device system a cardiac electrical signal episode sensed during a conduction system capture management test and determining capture test data from the cardiac electrical signal episode. The method may include comparing the capture test data to previous capture test data determined from a previous conduction system capture management test and generating an output based on the comparing of the capture test data to the previous capture test data. The method may include storing the output in memory.

Example 20. The method of example 19 further comprising detecting an alert condition of the conduction system capture management test corresponding to a change in the capture test data compared to the previous capture test data and displaying data in a user interface based on the generated output and the alert condition.

Example 21. The method of any of examples 19-20 further comprising determining the capture test data by identifying a number of different QRS waveform morphologies in the cardiac electrical signal episode.

Example 22. The method of example 21 further comprising detecting the alert condition by determining that the number of different QRS waveform morphologies in the cardiac electrical signal episode is different than a previous number of QRS waveform morphologies identified in a previous cardiac electrical signal episode sensed during a previous conduction system capture management test.

Example 23. The method of any of examples 21-22 further comprising determining the capture test data by determining a capture threshold for one or more of the different QRS morphologies identified in the cardiac electrical signal episode.

Example 24. The method of example 23 further comprising detecting an alert condition by determining that a capture threshold determined for at least one of the QRS morphologies identified in the cardiac electrical signal episode is different than a previously determined capture threshold determined for the respective QRS morphology identified in the previous cardiac electrical signal episode sensed during the previous capture management test.

Example 25. The method of any of examples 19-24 further comprising determining the capture test data by classifying at least one QRS waveform in the cardiac electrical signal episode according to a capture type.

Example 26. The method of any of examples 19-25 further comprising determining the capture test data by classifying a plurality of QRS waveforms in the cardiac electrical signal episode according to a plurality of capture types.

Example 27. The method of example 26 further comprising determining a capture threshold of one or more of the classified capture types of the plurality of capture types of the cardiac electrical signal episode.

Example 28. The method of example 27 further comprising detecting an alert condition by determining a change in the capture threshold determined for at least one of the classified capture types compared to a previous capture threshold determined for the at least one of the classified capture types in the previous conduction system capture management test.

Example 29. The method of any of examples 26-28 further comprising delivering conduction system pacing pulses at a plurality of pacing pulse outputs during the conduction system capture management test and during the previous conduction system capture management test. The method may further include determining an alert condition by determining that a classified capture type of the plurality of capture types occurs at a pacing pulse output of the plurality of pacing pulse outputs of the conduction system capture management test and that the classified capture type of the plurality of capture types does not occur at the pacing pulse output of the plurality of pacing pulse outputs of the previous conduction system capture management test.

Example 30. The method of any of examples 20-29 further comprising receiving, via the user interface, a user input labeling at least one QRS waveform of the cardiac electrical signal episode according to at least one of a morphology type or a capture type.

Example 31. The method of example 30 further comprising detecting an alert condition based on the capture test data determined from the cardiac electrical signal episode and the user input.

Example 32. The method of any of examples 30-31 further comprising adjusting by the processing circuitry the determination of capture test data from the cardiac electrical signal episode in response to receiving the user input.

Example 33. The method of example 32 further comprising adjusting the determination of capture test data by adjusting a criteria applied to a QRS morphology of the cardiac electrical signal episode for detecting a morphology change.

Example 34. The method of any of examples 32-33 further comprising adjusting the determination of capture test data by adjusting a criteria applied to a QRS morphology of the cardiac electrical signal episode for detecting a conduction system capture type.

Example 35. The method of any of examples 19-34 further comprising determining the capture test data by classifying each of a plurality of post-pace waveforms of the cardiac electrical signal episode according to a capture type as being one or more of: selective conduction system capture, non-selective conduction system capture, ventricular myocardial only capture without capture of the conduction system, left bundle branch capture, partial left bundle branch capture, right bundle branch capture, partial bundle branch capture, complete His bundle capture, partial His bundle capture or loss of capture.

Example 36. The method of any of examples 19-35 further comprising receiving the cardiac electrical signal episode transmitted from an implantable medical device, the cardiac electrical signal episode including at least one cardiac electrogram signal.

Example 37. Non-transitory computer-readable media storing instructions that, when executed by processing circuitry of a medical device system, cause the system to receive a cardiac electrical signal episode sensed during a conduction system capture management test, determine capture test data from the cardiac electrical signal episode, compare the capture test data to previous capture test data determined from a previous conduction system capture management test and generate an output based on the comparing of the capture test data to the previous capture test data, and store the output in memory of the medical device system.

Example 38. The non-transitory computer-readable media of example 37, wherein the instructions further cause the medical device system to detect an alert condition corresponding to a change in the capture test data compared to the previous capture test data and display data in the user interface based on the generated output and the alert condition.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram of a medical device system capable of delivering conduction system pacing (CSP) and sensing and analyzing cardiac electrical signals for determining capture test data according to some examples.

FIG. 2 is a conceptual diagram of an implantable medical device (IMD) connected to pacing and sensing leads for pacing a patient's heart and sensing cardiac electrical signals.

FIG. 3 is a conceptual diagram of an IMD coupled to a lead advanced to an alternative location within the heart for delivering CSP and sensing cardiac electrical signals.

FIG. 4 is a conceptual diagram of a leadless pacemaker positioned within the heart for providing CSP according to another example.

FIG. 5 is a conceptual diagram of the leadless pacemaker of FIG. 4 shown implanted in an alternative location for CSP.

FIG. 6 is a conceptual diagram of circuitry of an IMD configured to sense cardiac electrical signals and perform CSP according to some examples.

FIG. 7 is a flow chart of a method that may be performed by an IMD included in a medical device system according to some examples.

FIG. 8 is a conceptual diagram of a graphical user interface (GUI) that may be displayed to a user on an external device or a computing device to enable a user to program CM control parameters according to some examples.

FIG. 9 is a flow chart of a method that may be performed by processing circuitry of a medical device system, e.g., the medical device system of FIG. 1, for detecting an alert condition from a cardiac electrical signal episode recorded during a CM test according to some examples.

FIG. 10 is a flow chart of a method that may be performed by processing circuitry of a medical device system, e.g., the medical device system of FIG. 1, for detecting and responding to a change in capture test data according to another example.

FIG. 11 is a flow chart of a method for detecting an alert condition from a cardiac electrical signal episode by processing circuitry of a medical device system according to some examples.

FIG. 12 is a flow chart of a method for analyzing cardiac electrical signal episodes sensed during a CM test for detecting changes in capture test data by processing circuitry of a medical device system according to another example.

FIG. 13 is a diagram of a GUI that may be generated by medical device processing circuitry for displaying CM test data to a user according to some examples.

### DETAILED DESCRIPTION

A medical device system is described herein for obtaining, processing and analyzing cardiac electrical signal episodes sensed during CM tests for monitoring for changes in capture during CSP. A cardiac electrical signal episode, which may include one or more cardiac electrical signals sensed during a CM test, and/or data derived therefrom may be transmitted from an IMD for receipt by medical device system processing circuitry. The medical device system processing circuitry may be configured to detect changes in the cardiac electrical signal episode compared to a previous cardiac electrical signal episode sensed during a previous CM test. For example, the processing circuitry may determine capture test data from the cardiac electrical signal episode for detecting changes in the capture test data compared to a previous CM test. The processing circuitry may generate output based on the capture test data for display in a GUI. The generated output may be stored in memory of the medical device system and may be used in generating a display in a user interface based on the output. In some examples, an alert condition may be detected from the capture test data, e.g., based on a comparison to previous capture test data determined from a previous CM test.

As used herein, the term “conduction system pacing” (CSP) refers to delivery of one or more pacing pulses generated for delivery in the vicinity of a portion of the His-Purkinje conduction system of a heart. A CSP pulse may or may not capture the conduction system depending on the cathode and anode locations of a CSP electrode vector relative to the conduction system pacing site, the delivered pacing pulse energy and other factors. Complete or partial His bundle capture, complete or partial LBB capture and/or complete or partial RBB capture are non-limiting examples of CSP capture types. Complete or partial capture by a CSP pulse may be selective CSP capture when the CSP pulse captures the conduction system at the pacing site, completely or partially, without capturing ventricular myocardium. Capture of at least a portion of the conduction system is achieved when the pacing pulse energy delivered in a CSP pulse causes depolarization of tissue of the conduction system. The pacing-evoked depolarization arising at the CSP site can be propagated along the conduction system and further to the ventricular myocardium to cause depolarization of the ventricular myocardium and a subsequent, coordinated ventricular contraction. Non-selective CSP capture can occur when the CSP pulse energy is greater than the conduction system pacing capture threshold and the ventricular myocardial pacing capture threshold. Non-selective CSP capture by a CSP pulse causes depolarization of both a portion of the conduction system and ventricular myocardium. Ventricular myocardial only (VMO) capture may occur when the ventricular myocardium is captured (causing a pacing-evoked depolarization of the ventricular myocardium) without capturing any portion of the conduction system. A CSP pulse may fail to capture any portion of the conduction system resulting in a complete loss of capture (no pacing-evoked depolarization of the conduction system or the ventricular myocardium).

The medical device systems and techniques disclosed herein provide various improvements in a medical device system configured to generate and display various parameters determined from one or more cardiac electrical signals representative of the heart's electrical activity that a user may rely on when monitoring and assessing CSP effectiveness. The techniques disclosed herein improve the function of a medical device system in providing visual representations of CSP data useful in monitoring capture of CSP pulses.

The techniques disclosed herein therefore provide improvements in the computer-related field of cardiac monitoring and cardiac therapy delivery. By providing a medical device system capable of processing and analyzing cardiac electrical signals and displaying data in a GUI according to the techniques herein, the complexity and likelihood of human error in selecting CSP parameters for achieving capture of at least a portion of the conduction system or according to a desired capture type is reduced. Managing CSP capture in a patient using the techniques disclosed herein can improve or optimize the clinical benefit of CSP by reducing the burden on a clinician in identifying changes in CSP capture that may occur in a patient over time and simplifying the process of identifying changes in capture test data. The techniques disclosed herein may enable CSP to be delivered to a patient using techniques that promote maintaining capture of the conduction system in a manner that is simplified, flexible, and patient-specific for the managing clinician. The techniques disclosed herein may enable selection and programming of CM test control parameters and CSP pulse parameters for achieving conduction system capture and associated clinical benefits of CSP with a high degree of confidence.

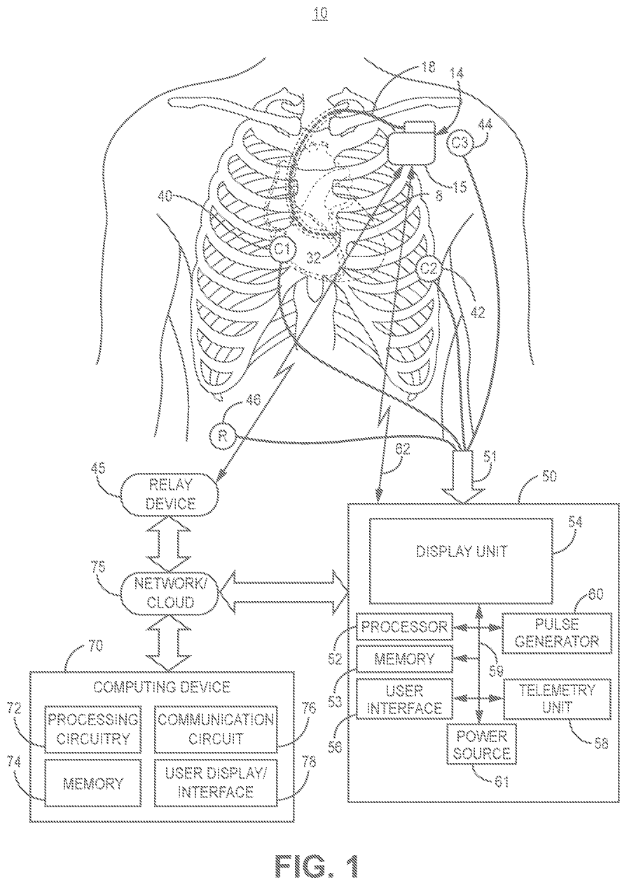

FIG. 1 is a conceptual diagram of a medical device system 10 capable of delivering CSP and sensing and analyzing cardiac electrical signals for determining capture test data. System 10 may include an IMD 14 coupled to at least one cardiac pacing and sensing lead 18 carrying one or more electrodes 32 for sensing cardiac electrical signals and delivering CSP. In FIG. 1, cardiac pacing lead 18 is shown advanced within a patient's heart 8 for positioning a pacing electrode 32 at a CSP site, e.g., within the interventricular septum at a CSP site in the area of the His bundle, the LBB or the RBB. It is to be understood, however, that a pacing electrode carried by a lead coupled to an IMD or a pacing electrode carried by a housing of a leadless IMD may be positioned at any desired CSP site and other examples of IMDs and CSP lead and/or electrode configurations are provided below.

The system 10 includes an external device 50, which can be configured for bi-directional communication with IMD 14, e.g., via wireless communication link 62. External device 50 may receive data from IMD 14 relating to CSP capture. CSP capture data can include a cardiac electrical signal episode, which may include one or more cardiac electrical signals sensed from respective sensing electrode vectors. The cardiac electrical signal episode or portions thereof may be displayed by external device 50, transmitted to another computing device 70, and/or processed and analyzed by external device 50 for detecting changes in CSP capture. CSP capture data received by external device 50 from IMD 14 may additionally or alternatively include parameters, metrics or other data determined by processing circuitry included in IMD 14 and transmitted to external device 50. As further described below, CSP capture data may include one or more QRS waveforms representing different QRS morphologies sensed during a CM test, one or more capture thresholds, and/or one or more morphology metrics determined from cardiac electrical signal(s) sensed by IMD 14. The CSP capture data may be obtained by IMD 14 from one or more cardiac signals sensed during a CM test.

External device 50 may be embodied as a patient monitor, programmer or pacing system analyzer used in a hospital, clinic or physician's office to acquire and analyze CSP capture data, e.g., a cardiac electrical signal episode sensed during a CM test. External device 50 may be a patient monitor located in a patient's home or other location for acquiring data from IMD 14. External device 50 may be a bedside or desktop device or a handheld device and may be a personal device such as a smartphone, tablet or other electronic device capable of receiving data wirelessly from IMD 14. In some examples, external device 50 may be a portable or home-based patient monitor included in a remote patient monitoring system such as the CARELINK™ monitoring system available from Medtronic, Inc., Dublin, Ireland.

In some examples, external device 50 may include an electrode/lead interface 51 for receiving input from skin or surface electrocardiogram (ECG) electrodes (shown conceptually as ECG electrodes 40, 42, 44 and 46). In some examples, ECG signals may be analyzed by processing circuitry of medical device system 10 in conjunction with CSP capture data received from IMD 14 for detecting changes in CSP capture according to the techniques disclosed herein.

External device 50 may include a processor 52, memory 53, display unit 54, user interface unit 56, telemetry unit 58, and power source 61. Power source 61 is coupled to the various units of external device 50 for providing power to circuits and components of external device 50 as needed. Power source 61 may include one or more rechargeable or non-rechargeable batteries or may be coupled to an external power source, such as plugged into an electrical outlet. In some examples, external device 50 may optionally include a pulse generator 60, for instance when external device 50 is a pacing system analyzer. Processor 52 may control pulse generator 60, when included, to generate pacing pulses for delivery as CSP pulses, e.g., via lead 18 prior to connection to IMD 14 in some examples, for testing or verifying implant sites and acceptable CSP capture thresholds.

Processor 52 may be coupled to other components and units of external device 50, e.g., via a data bus 59, for controlling the functions attributed to external device 50 herein. For example, processor 52 may pass ECG signals, CSP capture data received from IMD 14 and/or data derived therefrom to display unit 54 for displaying data in a GUI and/or to telemetry unit 58 for transmission to a computing device 70. External device telemetry unit 58 may be coupled to a communication network/cloud 75 for receiving and transmitting data to a computing device 70, which may be a personal computer, personal mobile device or other computing device at a remote location from the patient to enable remote monitoring of CSP capture data obtained from IMD 14 by a clinician or other user.

External device processor 52 executes instructions stored in memory 53. Processor 52 may include any one or more of a microprocessor, a controller, a digital signal processor (DSP), an application specific integrated circuit (ASIC), a field-programmable gate array (FPGA), or equivalent discrete or analog logic circuitry. In some examples, processor 52 may include multiple components, such as any combination of one or more microprocessors, one or more controllers, one or more DSPs, one or more ASICs, or one or more FPGAs, as well as other discrete or integrated logic circuitry. The functions attributed to processor 52 herein may be embodied as software, firmware, hardware or any combination thereof.

Memory 53 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media. Memory 53 may be configured to store instructions executed by processor 52 for obtaining and analyzing CSP capture data and displaying and/or transmitting CSP capture information according to the techniques disclosed herein. Memory 53 may store CSP capture data or information received from IMD 14 and/or determined by processor 52 for use generating a display of CSP capture information in a GUI on display unit 54 or for transmission to computing device 70.

Display unit 54, which may include a liquid crystal display, light emitting diodes (LEDs) and/or other visual display components, may generate a display of the CSP capture data and information, ECG signals, and/or EGM signals. Display unit 54 may generate a GUI including various windows, icons, user selectable menus, etc. to facilitate interaction by a user with external device 50. Display unit 54 may function as an input and/or output device using technologies including liquid crystal displays (LCD), quantum dot display, dot matrix displays, light emitting diode (LED) displays, organic light-emitting diode (OLED) displays, cathode ray tube displays, e-ink, or monochrome, color, or any other type of display capable of generating tactile, audio, and/or visual output. In some examples, display unit 54 is a presence-sensitive display that may serve as a user interface device that operates both as one or more input devices and one or more output devices.

In some examples, display unit 54 may generate a visual display of one or more ECG signal(s), EGM signal(s) and/or CSP capture data and/or other capture management information based on output generated by processing circuitry of the medical device system. The output may be stored in memory 53 for use in generating a GUI including data based on the generated output and any detected alert conditions. In other examples, display unit 54 may produce an output to a user in another fashion, such as via a sound card, video graphics adapter card, speaker, one or more USB interfaces, video and/or audio output interfaces, or any other type of device capable of generating audio, video, or other output. User interface unit 56 may include a data entry or pointing device such as a mouse, touch screen, keypad or the like, to enable a user to interact with external device 50 and a GUI displayed on display unit 54, e.g., to initiate and terminate a communication session, adjust settings of display unit 54, enter programmable control parameters for programming into IMD 14, or make other user requests.

Telemetry unit 58 may include a transceiver and antenna configured for bidirectional communication with a telemetry circuit included in IMD 14. Telemetry unit 58 is configured to operate in conjunction with processor 52 for sending and receiving data relating to IMD functions via a wireless communication link 62 with IMD 14. The communication link 62 may be established between IMD 14 and external device 50 using a radio frequency (RF) link such as BLUETOOTH®, Wi-Fi, Medical Implant Communication Service (MICS) or other RF or communication frequency bandwidth or communication protocols.

IMD 14 and/or external device 50 may be configured to communicate with one or more computing devices 70, e.g., over network/cloud 75. In some examples, medical device system 10 may include a communication relay device 45 for sending and receiving data between IMD 14 and network/cloud 75. Data retrieved from IMD 14, such as cardiac electrical signal episodes sensed during CM tests, may be analyzed by cloud-based algorithms for determining capture test data that may be transmitted to computing device 70 for presentation in a GUI for review by a clinician or other user. In some examples, the computing device(s) 70 can take the form of servers, personal computers, tablet computers or other computing devices associated with one or more healthcare providers (e.g., hospitals, medical data analytic companies, device manufacturers, etc.). Computing device 70 can collect data obtained by IMD 14 and/or the external device 50. In some examples, such data can be anonymized and aggregated to perform large-scale analysis e.g., using machine learning or other artificial intelligence (AI) techniques or other suitable data analysis techniques, to develop and improve capture detection algorithms using data collected by a large number of IMDs. Computing device 70 may transmit data to the external device 50 and/or IMD 14. For example, an updated algorithm for capture detection, CM test control parameters, updated CSP control parameters or other data used by IMD 14 for delivering CSP and monitoring CSP capture, e.g., by performing CM tests, can be provided to IMD 14, either directly or via relay device 45 or external device 50 via network/cloud 75.

Computing device 70 may include processing circuitry 72, memory 74, communication circuit 76 (e.g., components to facilitate wired or wireless communication with other devices either directly or via the network/cloud 75), and a user display/interface 78. Communication between computing device 70 and other devices can be performed via network 75, which can include the Internet, public and private intranet, a local or extended Wi-Fi network, cell towers, the plain old telephone system (POTS), direct wireless communication, etc. User display/interface 78 may include a computer monitor or display which can be a touch screen, a keypad, speakers, a camera, keyboard, mouse or other pointing device, as examples. CSP capture data and information may be received from IMD 14 and/or external device 50 or derived from data received from IMD 14, directly or indirectly, and/or external device 50 for display by computing device 70 on user display/interface 78. The clinician or other health care provider may view the CSP capture data and information and select CSP control parameters that may be programmed remotely into IMD 14 for controlling delivery of CSP based on analysis of capture test data derived from one or more cardiac electrical signal episodes sensed during CM test(s).

In some examples, as described below, a user may add labels or annotations to displayed CSP capture data or information, e.g., to label QRS waveforms in EGM or ECG signals. User entered labels may be received by the medical device system processing circuitry that verify or classify a QRS waveform following a CSP pulse according to a morphology type and/or a capture type. User entered labels may correct (relabel) an incorrect classification of a capture type made by processing circuitry of medical device system 10 in some instances. The processing circuitry of the medical device system 10, e.g., any combination of processing circuitry 72, processor 52, network/cloud based computing, and/or processing circuitry included in IMD 14 may analyze a cardiac electrical signal episode recorded during a CM test for detecting changes in CSP capture, e.g., relative to a previous CM test, using techniques as further described below.

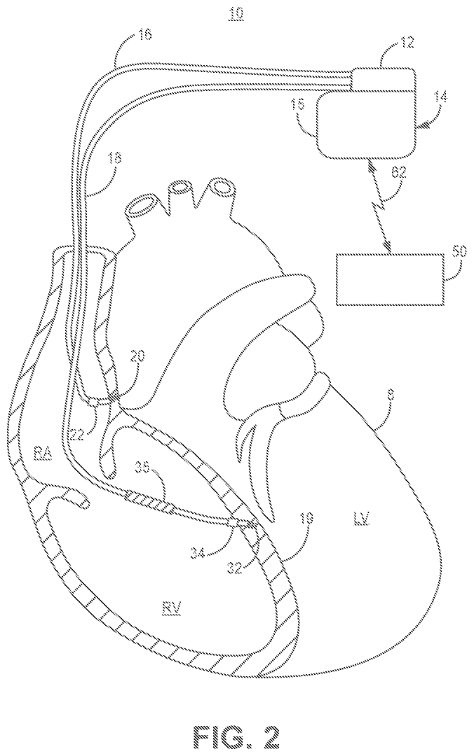

FIG. 2 is a conceptual diagram of IMD 14 connected to pacing and sensing leads 16 and 18 for pacing a patient's heart 8 and sensing cardiac electrical signals. IMD 14 is shown as a dual chamber device in FIG. 2, configured to receive a right atrial lead 16 positioned in the right atrium (RA) for delivering atrial pacing pulses and sensing atrial electrical signals (e.g., sensing atrial P-waves attendant to intrinsic atrial depolarizations) via electrodes 20 and 22. Lead 16 may be advanced transvenously to position electrodes 20 and 22 within the RA. IMD 14 may be configured to sense intrinsic atrial P-waves and deliver atrial pacing pulses in the absence of sensed P-waves using electrodes 20 and 22. Electrodes 20 and 22 can be electrically connected to IMD 14 via electrical conductors extending within the elongated lead body of lead 16 to a proximal lead connector (not shown) received by IMD connector assembly 12. IMD 14 may be configured to provide dual chamber sensing and pacing. For example, IMD 14 may deliver atrial synchronous ventricular pacing by setting an AV delay in response to each sensed P-wave or delivered atrial pacing pulse and deliver a CSP pulse via lead 18 upon the expiration of the AV delay to pace the ventricles in synchrony with the atria.

Lead 18 may be advanced transvenously into the RV via the RA for positioning pacing electrode 32 at a CSP site, e.g., within the inter-ventricular septum 19. Pacing electrode 32 can be referred to as a “tip electrode” because it is carried by CSP lead 18 at the distal lead tip. When pacing electrode 32 is advanced relatively superiorly within the inter-ventricular septum 19, pacing electrode 32 may be positioned along the inferior portion of the His bundle for delivering CSP. In other examples, pacing electrode 32 may be advanced within the inter-ventricular septum 19 in the vicinity of a bundle branch of the His-Purkinje system, e.g., at a LBB pacing site in the area of the LBB or at a RBB pacing site in the area of the RBB, for delivering CSP.

Pacing electrode 32 may be selected as a pacing cathode electrode in combination with ring electrode 34 as the return anode electrode for CSP. In some instances, the pacing pulse amplitude and pulse width (which may be referred to collectively as the “pacing pulse output”) may be selected to achieve cathodal capture at the cathode electrode for capturing at least at portion of one bundle branch. In other instances, the pacing pulse amplitude and pulse width may be selected to achieve cathodal and anodal capture, which may capture both the LBB and the RBB concurrently (by the same pacing pulse) to provide dual or bilateral bundle branch (BB) pacing using a single bipolar electrode pair. In other examples, either pacing electrode 32 or ring electrode 34 may be selected as the cathode electrode paired with IMD housing 15 in a unipolar pacing electrode vector.

Unipolar pacing may capture at least a portion of a single BB. In some cases, however, unipolar pacing may capture both the RBB and the LBB when a unipolar pacing pulse directly captures one bundle branch while virtual current or break excitation generated by the pacing electrode may excite the other bundle branch, potentially resulting in unipolar bilateral BB pacing, with capture of both the LBB and RBB.

While lead 18 is shown carrying one pacing and sensing electrode pair, pacing electrode 32 and ring electrode 34, it is to be understood that in other examples, lead 18 may include multiple pacing and sensing electrodes along its distal portion to provide one or more selectable bipolar pacing electrode vectors and/or one or more unipolar pacing electrode vectors (e.g., with housing 15) for delivering CSP pulses and sensing ventricular electrical signals.

Lead 18 may further include one or more cardioversion/defibrillation (CV/DF) electrodes 35 for delivering relatively high voltage shock therapies. A CV/DF electrode generally has a high surface area and may be an elongated coil electrode as illustrated by coil electrode 35 on lead 18. In addition to delivering relatively low voltage atrial and CSP pulses, IMD 14 can be configured as an implantable cardioverter defibrillator (ICD) capable of delivering high voltage shock therapies for terminating ventricular tachycardia or fibrillation. Coil electrode 35 may also be used in sensing electrode vectors, e.g., with either of pacing electrode 32 or ring electrode 34, for sensing a ventricular EGM signal that may be transmitted to external device 50 via communication link. Other examples of pacing lead configurations for delivering CSP that may be used in conjunction with the techniques described herein are generally disclosed in U.S. Publication No. 2022/0023640 (Zhou, et al.) and in U.S. Pat. No. 11,207,529 (Zhou), both of which are incorporated herein by reference in their entirety.

Electrodes 20, 22, 32, 34, and 35 (and any other electrodes shown or described herein) may be, without limitation, titanium, platinum, iridium or alloys thereof and may include a low polarizing coating, such as titanium nitride, iridium oxide, ruthenium oxide, platinum black, among others. Electrodes 20 and 32 are shown as helical screw in electrodes that may serve as fixation members for securing the distal ends of respective leads 16 and 18 at a desired pacing and sensing site. In other examples, either of leads 16 or 18 may include a fixation member that includes one or more tines, hooks, barbs, helices or other fixation member(s) that anchor the distal end of the lead 16 or 18 at the implant site. In some examples, electrodes 20 and 32 may be other types of tissue-piercing electrodes, such as fishhook or straight electrodes with a tissue-piercing distal tip.

Electrodes 22 and 34 are each shown as ring electrodes circumscribing the elongated lead body of the respective lead 16 or 18, at a location proximal from the respective tip electrode 20 or 32. In other examples, electrodes used for sensing and pacing in a medical device system configured to deliver CSP may include button, spherical, segmented, or other types of electrodes.

IMD 14 includes a housing 15 that encloses electronic circuitry configured to perform cardiac signal sensing and therapy delivery functions attributed to IMD 14.

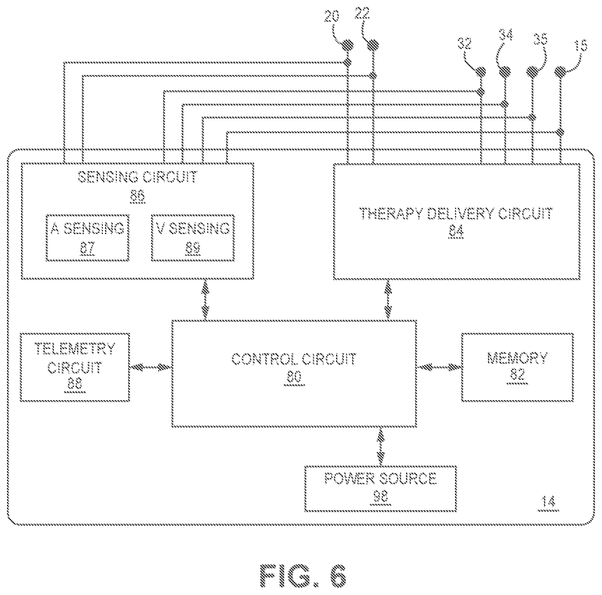

Examples of circuitry that may be included in IMD 14 are described below in conjunction with FIG. 6. IMD 14 includes a connector assembly 12, sometimes called a “connector block” or “header,” having connector bores for receiving proximal lead connectors (not seen in FIG. 2) of each of the respective leads 16 and 18 coupled to IMD 14. Each of leads 16 and 18 include insulated electrical conductors (not shown) extending through one or more lumens within the elongated, electrically insulating lead bodies of respective leads 16 and 18. Each electrical conductor extends from a respective electrode 20, 22, 32, 34 and 35 to the proximal lead connector of the corresponding lead 16 or 18 to provide electrical connection to electrical contacts within connector assembly 12. Electrical connection of the electrodes 20, 22, 32, 34 and 35 to internal electronic circuitry of IMD 14 is provided by electrical feedthroughs in connector assembly 12 that cross the hermetically sealed housing 15 of IMD 14.

In this way, the insulated electrical conductors extending through leads 16 and 18 carry electrical signals from therapy delivery circuitry within housing 15 to electrodes 20, 22, 32, 34 and/or 35 for delivering electrical stimulation pulses. The insulated electrical conductors can carry cardiac electrical signals from heart 8 via electrodes 20, 22, 32, 34 and/or 35 to sensing circuitry within housing 15 for obtaining atrial and ventricular EGM signals. As described above, IMD 14 may communicate via wireless telemetry with external device 50 and/or one or more computing devices 70. For instance, external device 50 may receive EGM signals, delivered pacing pulse marker signals, and/or other CSP capture data that are transmitted by IMD 14 for use in detecting changes in CSP capture by processing circuitry of external device 50, network/cloud 75, and/or computing device 70 for display to a clinician or other user.

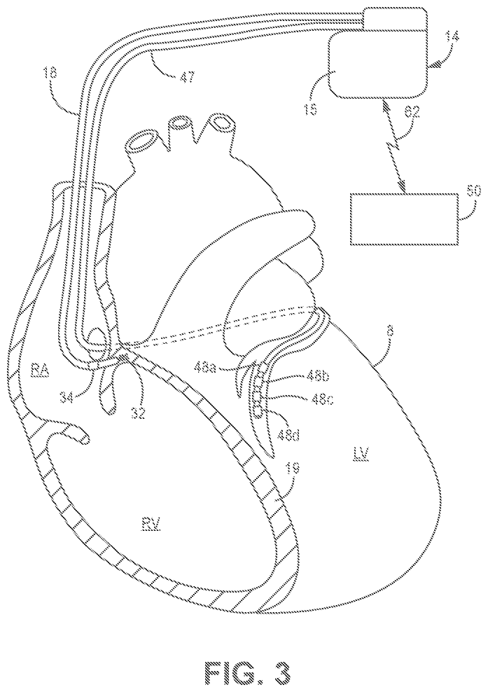

FIG. 3 is a conceptual diagram of IMD 14 coupled to lead 18 advanced to an alternative location within the heart 8 for delivering CSP and sensing cardiac electrical signals. In this example, the distal portion of lead 18 is advanced within the RA for sensing ventricular electrical signals and delivering CSP pulses to or in the vicinity of the His bundle from a right atrial approach. The pacing tip electrode 32 of lead 18 can be advanced into the cardiac tissue in the area of the His bundle, e.g., between the His bundle and the coronary sinus and adjacent the tricuspid valve. A target entry site for electrode 32 may correspond to or lie within the Triangle of Koch in some examples for achieving CSP at a His bundle pacing site. Pacing electrode 32 may be paired with the return anode ring electrode 34 for delivering CSP pulses and for sensing raw cardiac electrical signals, which may be processed for obtaining a ventricular EGM signal.

In some examples, CSP may be delivered in combination with myocardial pacing of the left ventricle (LV) that can be delivered via a left ventricular lead 47 for further improvement in electrical and mechanical synchrony of the RV and LV, e.g., during cardiac resynchronization therapy (CRT). left ventricular lead 47 may be advanced into the RA, through the coronary sinus ostium and into a cardiac vein of the LV for positioning electrodes 48a, 48b, 48c and 48d (collectively “LV electrodes 48”) along the left ventricular myocardium for sensing ventricular electrical signals and pacing the LV myocardium. Left ventricular lead 47 is shown as a quadripolar lead carrying four electrodes 48a-d that may be selected in various bipolar pacing electrode pairs for pacing the myocardial tissue of the LV and for sensing LV signals. One of LV electrodes 48 may be selected in combination with IMD housing 15 for delivering unipolar LV myocardial pacing in some instances and/or for sensing ventricular EGM signals that may be transmitted to external device 50 via communication link 62. Left ventricular lead 47 may have more or fewer electrodes than the four electrodes 48 shown in FIG. 3.

When lead 18 is positioned for delivering CSP, CSP may be combined with ventricular myocardial pacing using left ventricular lead 47 to correct a ventricular conduction delay and achieve electrical and mechanical synchrony of the ventricles. As such, in some examples, IMD 14 may control CSP pulse delivery in combination with LV myocardial pacing pulse delivery at specified time intervals which may include an AV delay and/or a ventricular-to-ventricular (VV) delay. The AV delay may control the timing of the CSP pulses and/or the LV myocardial pacing pulses relative to an atrial event, e.g., sensed P-wave or delivered atrial pacing pulse. (e.g., when RA lead 16 is coupled to IMD 14 as shown in FIG. 2). In some examples, a VV delay may control the timing between a CSP pulse delivered via lead 18 and an LV myocardial pacing pulse delivered via LV lead 47.

It is to be understood that left ventricular lead 47 is optional. In some examples, IMD 14 is coupled only to lead 18 advanced into the RA or the RV for positioning at least one electrode 32 at a CSP site for delivering CSP and sensing ventricular EGM signals. In other examples, RA lead 16 as shown in FIG. 2 is implanted in combination with lead 18 for delivering CSP in a dual chamber sensing and pacing system. In still other examples, IMD 14 may be a multi-chamber device configured to receive lead 16, lead 18 and lead 47 for delivering cardiac pacing pulses to the RA, His-Purkinje conduction system, and LV myocardium as needed. External device 50 (or relay device 45) may receive one or more EGM signals from IMD 14 sensed using any available EGM sensing electrode vector. The one or more EGM signals may be sensed during a CM test and transmitted as a cardiac electrical signal episode for analysis by processing circuitry of the medical device system for detecting changes in CSP capture.

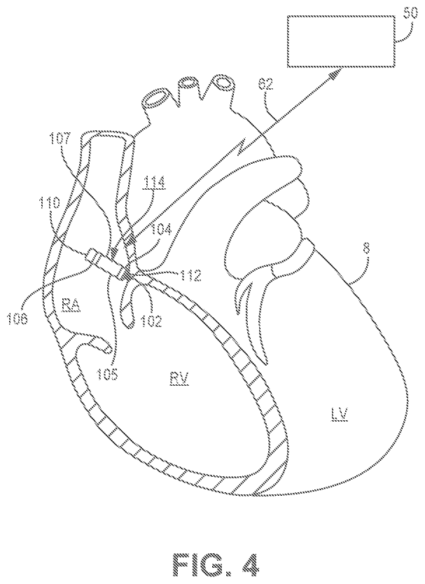

FIG. 4 is a conceptual diagram of a leadless pacemaker 114 positioned within the RA for providing CSP according to another example. Pacemaker 114 may include a distal tip electrode 102 located on and extending from a distal end 112 of the pacemaker housing 105. Pacemaker 114 is shown implanted in the RA of the patient's heart 8 to place distal tip electrode 102 for delivering CSP pulses in the area of the His bundle. For example, the distal tip electrode 102 may be inserted into the inferior end of the interatrial septum, beneath the AV node and near the tricuspid valve annulus to position tip electrode 102 in, along or proximate to the His bundle. Distal tip electrode 102 may be a tissue piercing electrode and is a helical electrode providing fixation to anchor the pacemaker 114 at the implant position in some examples. In other examples, pacemaker 114 may include a fixation member that includes one or more tines, hooks, barbs, helices or other fixation member(s) that anchor the distal end of the pacemaker 114 at the implant site. A portion of the distal tip electrode 102 may be electrically insulated such that only the most distal end of tip electrode 102, furthest from housing distal end 112, is exposed to provide targeted pacing at a CSP site.

One or more additional housing-based electrodes 104 and 106 may be carried on the surface of the housing 105 of pacemaker 114 proximal to distal tip electrode 102. Electrodes 104 and 106 are shown as ring electrodes circumscribing the longitudinal sidewall 107 of pacemaker housing 105, which may be generally cylindrical in shape. Longitudinal sidewall 107 extends from distal end 112 to proximal end 110 of housing 105. One of electrodes 104 or 106 may serve as a return anode electrode in combination with the cathode tip electrode 102 for pacing and sensing. For example, pacing of the conduction system may be achieved using the distal tip electrode 102 as the cathode electrode and either of the housing-based electrodes 104 and 106 as the return anode. In other examples, a return anode electrode used in sensing and pacing may be positioned on housing proximal end 110.

Cardiac electrical signals may be sensed by pacemaker 114 using a sensing electrode pair selected from electrodes 102, 104 and 106. For example, a cardiac electrical signal may be sensed using distal tip electrode 102 and distal housing-based electrode 104 or proximal housing-based electrode 106. A second cardiac electrical signal may be sensed using electrodes 104 and 106. In some examples, atrial P-waves may be sensed from a signal received via electrodes 104 and 106 and/or atrial pacing pulses may be delivered via electrodes 104 and 106. Atrial synchronous CSP pulses may be delivered via electrodes 102 and 104 at an AV delay following sensed atrial P-waves and/or delivered atrial pacing pulses. An EGM signal sensed by pacemaker 114 and/or CSP capture data derived therefrom may be transmitted to external device 50 via communication link 62 (or to a computing device 70 via network/cloud 75 shown in FIG. 1).

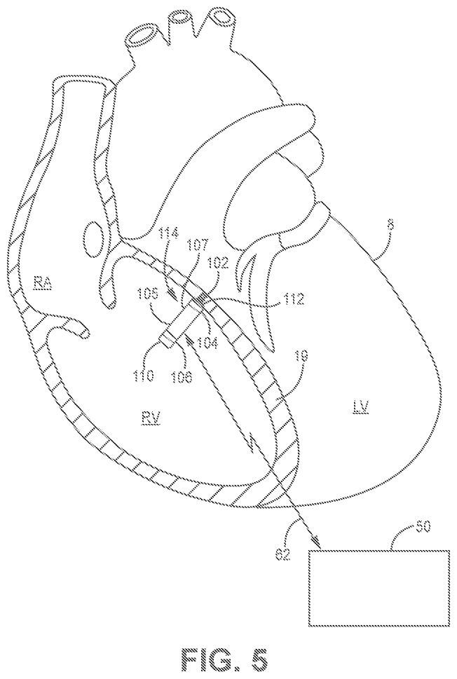

FIG. 5 is a conceptual diagram of the leadless pacemaker 114 of FIG. 4 shown implanted in an alternative location for CSP. Pacemaker 114 may be implanted within the RV along the inter-ventricular septum 19 for providing CSP in some examples. Techniques disclosed herein may be used in conjunction with a leadless pacemaker, such as pacemaker 114, having a pacing electrode 102 coupled to and extending directly from the pacemaker housing 105, without requiring an intervening medical lead coupled to the pacemaker 114 for carrying the pacing and sensing electrode(s).

In this example, pacemaker 114 may be positioned within the RV for advancing the pacing tip electrode 102 extending from the distal end 112 of pacemaker housing 105 into the inter-ventricular septum 19 for delivering CSP, e.g., in the area of an inferior portion of the His bundle or along one or both of the RBB and LBB depending on the relative positioning of distal tip electrode 102. Distal tip electrode 102 is shown as a “screw-in” helical electrode but may be configured as other types of tissue-piercing electrodes capable of being advanced within the septal tissue. A proximal portion of the distal tip electrode 102 may be electrically insulated, e.g., with a coating, such that only a distal portion of tip electrode 102, furthest from pacemaker housing distal end 112, is exposed to provide targeted pacing at a tissue site that includes the His bundle, LBB and/or RBB.

In other examples, distal tip electrode 102 may be formed having a straight shaft with a distal active electrode portion or other type of electrode, which may be a tissue-piercing electrode that is advanceable through the inter-ventricular septum 19 to deliver CSP, e.g., in a left portion of the septum 19 in the area of the LBB. In some examples, pacemaker 114 may include a fixation member that includes one or more tines, hooks, barbs, helices or other fixation member(s) that anchor the distal end 112 of the pacemaker 114 at the implant site and may not function as an electrode. Examples of leadless intracardiac pacemakers that may be configured for delivering cardiac pacing pulses to the conduction system that may be used in conjunction with the techniques described herein are generally disclosed in the above-incorporated U.S. Pat. No. 11,207,529 (Zhou) and in U.S. Publication No. 2019/0083800 (Yang, et al.), incorporated herein by reference in its entirety.

Pacemaker 114 may include the distal housing-based ring electrode 104 along or near the distal end 112 of pacemaker housing 105. In an example, distal housing-based ring electrode 104 may be selectable as the return anode electrode with distal tip electrode 102 for bipolar pacing of the LBB and/or RBB in the vicinity of the distal tip electrode 102. Bipolar bilateral BB pacing of both the RBB and LBB simultaneously may be achieved by cathodal capture of the LBB at distal tip electrode 102 and anodal capture of the RBB by distal ring electrode 104. The polarities of the distal tip electrode 102 and the distal ring electrode 104 may be reversed to achieve cathodal capture of the RBB and anodal capture of the LBB in some examples. Distal ring electrode 104 is shown as a ring electrode circumscribing a distal portion of the housing 105 but may alternatively be a distal housing-based electrode in the form of a button electrode, hemispherical electrode, segmented electrode or the like and may be along the face of distal end 112 of housing 105 and/or along longitudinal sidewall 107.

In the example shown, a housing-based proximal ring electrode 106, which may circumscribe all or a portion of the longitudinal sidewall 107 of the housing 105, may be provided as a return anode electrode. In other examples, a return anode electrode used in sensing and pacing may be positioned on housing proximal end 110 and may be a button, ring or other type of electrode. CSP in the area of the LBB may be achieved using the tip electrode 102 as the cathode electrode and the proximal ring electrode 106 as the return anode. CSP in the area of the RBB and/or myocardial tissue of inter-ventricular septum 19 may be achieved using the distal ring electrode 104 as a cathode electrode and the proximal ring electrode 106 as the return anode. In this way, bilateral or dual bundle branch pacing of the conduction system may be achieved using two different bipolar pacing electrode vectors carried by housing 105.

Cardiac electrical signals produced by heart 8 may be sensed by pacemaker 114 using electrodes 102, 104 and/or 106. The cardiac electrical signal received via electrodes 102 and 104, electrodes 104 and 106 and/or electrodes 102 and 106, for example, may be sensed by pacemaker 114 and processed by processing circuitry of IMD 14 and/or transmitted wirelessly, e.g., as EGM signals, to external device 50 via communication link 62 or to computing device 70 via network/cloud 75 (shown in FIG. 1). The EGM signals may then be displayed and/or further processed and analyzed, e.g., by the processor 52 of external device 50, processing circuitry 72 of computing device 70 or by cloud based computing on network/cloud 75, for providing a user with visual representations of sensed EGM signals and/or CSP capture data.

The examples of FIGS. 1-5 present various lead and/or electrode configurations that may be implemented for delivering CSP in a medical device system configured to perform the techniques disclosed herein for analyzing cardiac electrical signals and generating CSP capture data. The various lead and electrode configurations described and shown in the accompanying drawings are intended to be illustrative in nature. It is to be understood that the leads and electrodes illustrated in FIGS. 1-5 may be implanted in different combinations and/or other locations than the examples shown and some leads and/or electrodes may be omitted or additional leads and/or electrodes may be provided in a medical device system configured to deliver CSP and monitor CSP capture. In some examples, a leadless IMD, e.g., pacemaker 114, may be implanted in a patient for CSP in combination with another implanted IMD, e.g., an IMD connected to a RA lead for pacing and sensing in the right atrium and/or an ICD coupled to transvenous or non-transvenous extracardiac leads for providing tachyarrhythmia detection and therapy delivery. A variety of lead-based and leadless IMD and electrode configurations may be conceived for sensing cardiac electrical signals and delivering CSP pulses which may be used in conjunction with the techniques disclosed herein for analyzing cardiac electrical signals and presenting CSP capture data to a user, e.g., in GUI displayed by external device 50 and/or by user display/interface 78 of computing device 70.

FIG. 6 is a conceptual diagram of circuitry of an IMD configured to sense cardiac electrical signals and perform CSP according to some examples. The diagram of FIG. 6 is described with reference to IMD 14 coupled to electrodes 20 and 22 carried by RA lead 16 and electrodes 32, 34 and 35 carried by lead 18 as shown in FIG. 2, as an illustrative example. It is to be understood, however, that the functionality attributed to the various circuits and components shown in FIG. 6 for sensing cardiac signals and delivering CSP may be implemented in conjunction with other lead and electrode configurations, including the leadless pacemaker 114 of FIGS. 4 and 5 or other medical devices configured to deliver CSP pulses and sense cardiac electrical signals.

Housing 15 is represented as an electrode in FIG. 6 for use in cardiac electrical signal sensing and, in some examples, for delivery of unipolar pacing pulses. When IMD 14 is implemented as an ICD, housing 15 may be used as an active can electrode for delivery of CV/DF shock pulses. The electronic circuitry enclosed within housing 15 includes software, firmware and hardware that cooperatively monitor cardiac electrical signals, determine when a pacing pulse is necessary, and deliver electrical pacing pulses to the patient's heart as needed according to a programmed pacing mode and pacing pulse control parameters. The electronic circuitry can include a control circuit 80, memory 82, therapy delivery circuit 84, cardiac electrical signal sensing circuit (also referred to herein as “sensing circuit”) 86, telemetry circuit 88, and power source 98.

Power source 98 provides power to the circuitry of IMD 14 including components of circuits 80, 82, 84, 86, and 88 as needed. Power source 98 may include one or more energy storage devices, such as one or more rechargeable or non-rechargeable batteries. The connections between power source 98 and components of circuits 80, 82, 84, 86, and 88 are to be understood from the general block diagram of FIG. 6 but are not shown for the sake of clarity. For example, power source 98 may be coupled to one or more charging circuits included in therapy delivery circuit 84 for providing the power needed to charge holding capacitors included in therapy delivery circuit 84 that are discharged at appropriate times under the control of control circuit 80 for delivering electrical stimulation pulses. Power source 98 is also coupled to components of sensing circuit 86, such as sense amplifiers, analog-to-digital converters, switching circuitry, etc. as needed for sensing cardiac electrical signals. Power source 98 may provide power to the various components and circuits of telemetry circuit 88 and memory 82 as needed, which may be under the control of control circuit 80.

The circuits shown in FIG. 6 represent functionality included in IMD 14 and may include any discrete and/or integrated electronic circuit components that implement analog and/or digital circuits capable of producing the functions attributed to IMD 14 (or pacemaker 114) herein. The various components may include an application specific integrated circuit (ASIC), an electronic circuit, a processor (shared, dedicated, or group) and memory that execute one or more software or firmware programs, a combinational logic circuit, state machine, or other suitable components or combinations of components that provide the described functionality. Providing software, hardware, and/or firmware to accomplish the described functionality in the context of any modern medical device system, given the disclosure herein, is within the abilities of one of skill in the art.

Control circuit 80 communicates, e.g., via a data bus, with therapy delivery circuit 84 and sensing circuit 86 for cooperatively sensing cardiac electrical signals and controlling delivery of cardiac electrical stimulation therapies in response to sensed cardiac event signals, e.g., P-waves attendant to atrial depolarizations and R-waves attendant to ventricular depolarizations, or the absence thereof. The available electrodes may be selectively coupled, e.g., via switching circuitry, to therapy delivery circuit 84 for delivering electrical stimulation pulses and/or to sensing circuit 86 for sensing cardiac electrical signals produced by the heart. Sensed cardiac electrical signals may include both intrinsic signals (such as intrinsic P-waves and intrinsic R-waves) produced by the heart in the absence of a pacing pulse that captures the heart and evoked response signals following a delivered pacing pulse of sufficient energy to cause capture of cardiac tissue.

Sensing circuit 86 may include one or more sensing channels for receiving raw cardiac electrical signals from one or more sensing electrode vectors. For example, an atrial signal may be sensed using right atrial lead electrodes 20 and 22 coupled to atrial sensing (A sensing) channel 87. A ventricular signal may be sensed by ventricular sensing (V sensing) channel 89 using electrodes 32, 34 and/or 35 carried by lead 18. In some examples, V sensing channel 89 may include multiple ventricular sensing channels for receiving raw signals from multiple sensing electrode vectors that may include at least one electrode in or proximate to the ventricular chambers. For instance, V sensing channel 89 may include a near field sensing channel for receiving a raw near field signal using electrodes 32 and 34 of lead 18 in a bipolar sensing electrode pair. V sensing channel 89 may include a far field or unipolar sensing channel for receiving a raw far field signal. For example, a raw far field signal may be received using a second electrode vector having electrodes spaced further apart than the electrodes of the near field sensing electrode vector. A far field signal may be sensed, for example, using pacing electrode 32 or ring electrode 34 of lead 18 paired with IMD housing 15. In some examples, V sensing channel 89 may receive a raw far field signal sensed using pacing electrode 32 or ring electrode 34 paired with coil electrode 35. In other examples, a far field signal may be sensed using coil electrode 35 paired with IMD housing 15.

Sensing circuit 86 may include switching circuitry for selectively coupling a sensing electrode pair from the available electrodes to a respective sensing channel of A sensing channel 87 or V sensing channel 89. Switching circuitry may include a switch array, switch matrix, multiplexer, or any other type of switching device suitable to selectively couple components of sensing circuit 86 to selected electrodes.

Each of the sensing channels 87 and 89 of sensing circuit 86 may include an input filter for receiving a raw cardiac electrical signal from a respective pair of sensing electrodes, a pre-amplifier, an analog-to-digital converter (ADC), and a bandpass filter for producing a multi-bit digital cardiac electrical signal, which may be referred to as an “intracardiac EGM” signal when the raw signal is sensed using at least one electrode within a heart chamber. A multi-bit EGM signal may be passed from sensing circuit 86 to control circuit 80 for processing and analysis and/or for transmission to external device 50, network/cloud 75 and/or computing device 70 (e.g., shown in FIG. 1) for processing and analysis and/or display.

Each sensing channel 87 and 89 may include cardiac event detection circuitry, which may include one or more sense amplifiers, filters, rectifiers, threshold detectors, comparators, analog-to-digital converters (ADCs), timers or other analog or digital components, for detecting cardiac electrical event signals. For example, an atrial event detector may be included in A sensing channel 87 for sensing intrinsic P-waves attendant to intrinsic atrial depolarizations using one or both of electrodes 20 and 22 carried by right atrial lead 16. A ventricular event detector may be included in V sensing channel 89 for sensing intrinsic R-waves attendant to intrinsic ventricular depolarizations using electrodes 32 and 34 carried by lead 18.

A cardiac event sensing threshold, such as a P-wave sensing threshold and an R-wave sensing threshold, may be automatically adjusted by sensing circuit 86 under the control of control circuit 80, e.g., based on timing intervals and sensing threshold values determined by control circuit 80, stored in memory 82, and/or controlled by hardware, firmware and/or software of control circuit 80 and/or sensing circuit 86. The R-wave sensing threshold, for example, may be controlled to start at a starting threshold voltage following a post-ventricular blanking period then decrease according to one or more decay rates and/or one or more stepwise decrements until reaching a minimum sensing threshold. The minimum R-wave sensing threshold may be set to a programmed sensitivity of the R-wave detection circuitry. The sensitivity, programmed to a voltage level, typically in millivolts, is the lowest voltage level above which a cardiac event signal, e.g., a P-wave or an R-wave, can be sensed by the cardiac event detection circuitry of the respective A sensing channel 87 or V sensing channel 89.

Upon detecting a cardiac electrical event signal based on a sensing threshold crossing, sensing circuit 86 may produce a sensed event signal that is passed to control circuit 80. For example, an atrial event detector may produce an atrial sensed event signal in response to a P-wave sensing threshold crossing. A ventricular event detector may produce a ventricular sensed event signal in response to an R-wave sensing threshold crossing. The sensed event signals can be used by control circuit 80 for starting pacing escape interval timers that control the basic time intervals used for scheduling cardiac pacing pulses, e.g., atrial pacing pulses and CSP pulses and, in some cases, LV myocardial pulses.

Control circuit 80 may include various timers or counters for counting down an AV delay, a VV delay, an atrial pacing lower rate interval, a ventricular pacing lower rate interval, or other pacing escape intervals according to a pacing mode and pacing control parameters. A sensed event signal may trigger or inhibit a pacing pulse depending on the particular programmed pacing mode. For example, a P-wave sensed event signal received from sensing circuit 86 may cause control circuit 80 to inhibit a scheduled atrial pacing pulse and schedule a CSP pulse at an AV delay. If the AV delay expires before control circuit 80 receives an R-wave sensed event signal from sensing circuit 86, therapy delivery circuit 84 may generate and deliver a CSP pulse at the AV delay following the sensed P-wave and in this way deliver atrial-synchronized ventricular pacing. If an R-wave sensed event signal is received from sensing circuit 86 before the AV delay expires, the scheduled CSP pulse may be inhibited. The AV delay controls the amount of time between an atrial event, paced or sensed, and a CSP pulse to promote electrical and mechanical synchrony of the heart chambers.

In other instances, a ventricular pacing lower rate interval (LRI) may be set by control circuit 80 to schedule a CSP pulse following a delivered CSP pulse or sensed R-wave. The LRI may correspond to a programmed ventricular lower rate or may be adjusted to a temporary LRI by control circuit 80 to deliver rate response pacing when an increase in patient activity level is detected, e.g., by an accelerometer signal or other patient activity sensor included in IMD 14 (not shown in FIG. 6). When IMD 14 is operating in a dual chamber pacing mode, e.g., a DDD mode, when a P-wave is sensed during the LRI, a CSP pulse can be triggered to occur at the AV delay, and the LRI can restarted upon delivery of the CSP pulse. If the LRI expires without a sensed P-wave or a sensed R-wave, the CSP pulse can be delivered at the expiration of the LRI, and the LRI can be restarted. Control circuit 80 may be configured to control therapy delivery circuit 84 to deliver CSP pulses according to a variety of pacing modes and pacing therapies, which may include bradycardia pacing, post-shock pacing, anti-tachycardia pacing (ATP), cardiac resynchronization therapy (CRT), rate response pacing, etc.

Therapy delivery circuit 84 may include charging circuitry, one or more charge storage devices such as one or more holding capacitors, an output capacitor, and switching circuitry that controls when the holding capacitor(s) are charged and discharged across the output capacitor to deliver a pacing pulse to a selected pacing electrode vector coupled to the therapy delivery circuit 84. Therapy delivery circuit 84 may include one or more pacing channels. In the example of IMD 14, therapy delivery circuit 84 may include an atrial pacing channel and a ventricular pacing channel each including one or more holding capacitors, one or more switches, and an output capacitor for producing pacing pulses delivered by the respective RA lead 16 (e.g., via electrodes 20 and 22) or lead 18 (e.g., via electrodes 32 and 34). In other examples, the atrial and ventricular pacing pulses may be generated and delivered by shared pulse generating circuitry.

Charging of a holding capacitor to a programmed pacing voltage amplitude and discharging of the capacitor for a programmed pacing pulse width may be performed by therapy delivery circuit 84 according to control signals received from control circuit 80. For example, a pace timing circuit included in control circuit 80 may include programmable digital counters set by a microprocessor of the control circuit 80 for controlling the basic pacing time intervals associated with various single chamber and/or dual chamber pacing modes, multi-chamber pacing modes when LV lead 47 (shown in FIG. 3) is connected to IMD 14 for delivering CRT, and/or for delivering ATP sequences, as examples. The microprocessor of control circuit 80 may also set the amplitude, pulse width, polarity or other characteristics of the cardiac pacing pulses, which may be based on programmed values stored in memory 82.

IMD 14 may be configured to detect non-sinus tachycardia and deliver ATP. When IMD 14 is configured as an ICD for detecting tachyarrhythmia and delivering CV/DF shocks, therapy delivery circuit 84 may include high voltage therapy delivery circuitry for generating high voltage shock pulses in addition to low voltage therapy circuitry for generating low voltage pacing pulses. In response to detecting ventricular tachycardia or fibrillation, control circuit 80 may control therapy delivery circuit 84 to deliver a CV/DF shock. The high voltage therapy circuitry may include high voltage capacitors and high voltage charging circuitry for generating and delivering CV/DF shock pulses using elongated coil electrodes, e.g., coil electrode 35, carried by one or more leads coupled to IMD 14 and/or housing 15.

Control parameters utilized by control circuit 80 for sensing cardiac event signals (e.g., P-waves and R-waves) and controlling pacing therapy delivery may be programmed into memory 82 via telemetry circuit 88. Telemetry circuit 88 may include a transceiver and antenna for communicating with external device 50 (e.g., shown in FIG. 1) using radio frequency communication or other communication protocols as described above. Under the control of control circuit 80, telemetry circuit 88 may receive downlink telemetry from and send uplink telemetry to the external device 50 or computing device 70. In some cases, telemetry circuit 88 may be used to transmit and receive communication signals to/from another medical device implanted in the patient. Telemetry circuit 88 can transmit EGM signals, pacing pulse timing markers, atrial and ventricular sensed event signal markers and other sensing and pacing related data for receipt by external device 50 in real time and/or from stored EGM signal episodes, which may be displayed by external device 50 or computing device 70.

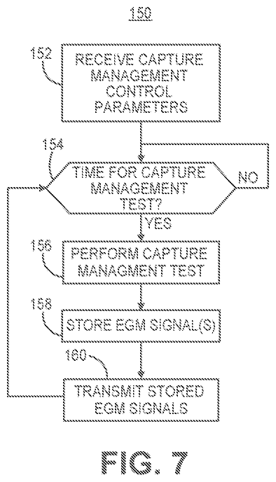

FIG. 7 is a flow chart 150 of a method that may be performed by an IMD included in a medical device system according to some examples. For the sake of illustration, the process of flow chart 150 and other flow charts presented herein are described in conjunction with IMD 14 performing functions attributed to an implantable device performing a CM test. It is to be understood that another IMD configured to deliver CSP and sense at least one cardiac electrical signal may perform the functions attributed to the implantable device in the techniques presented herein. Furthermore, in some example, it is possible that one IMD may be delivering CSP while a second IMD and/or external device senses and stores cardiac electrical signals, e.g., while the first IMD is delivering CSP pulses according to a CM test protocol. As such, the process of flow chart 150 could be performed cooperatively by more than one medical device, e.g., including at least one IMD configured to deliver CSP pulses and one or more second devices, implantable and/or external, configured to sense and store cardiac electrical signal(s), which may be EGM and/or ECG signals, recorded as a cardiac electrical signal episode during a CM test.

At block 152, IMD 14 receives CM control parameters. In some examples, CM control parameters used by IMD 14 for controlling the time that CM tests are performed and/or how the CM test is performed may be programmable by a user to tailor the CM test to an individual patient's needs. In some examples, programmable CM control parameters are used by IMD 14 for selecting a pacing output based on a CM test.

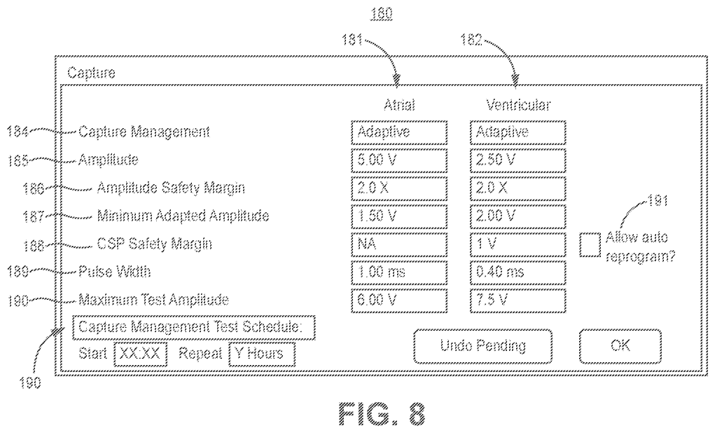

FIG. 8 is a conceptual diagram 180 of a GUI that may be displayed to a user on external device 50 or by computing device 70 to enable a user to program CM control parameters according to some examples. The diagram 180 may represent a screenshot or window of a CM programming screen that can be displayed by display unit 54 of external device 50 or by user display/interface 78 of computing device 70. A user may select the CM programming screen to program control parameters used by IMD 14 for performing a CM test and, in some cases, for making automatic programming adjustments subsequent to a CM test.

In this example, IMD 14 is a dual chamber device such that the CM programming screen includes an atrial channel 181 of CM control parameters and a ventricular channel 182 of CM control parameters. Each of the atrial channel 181 and the ventricular channel 182 may include a programmable capture management mode 184, a pacing pulse amplitude 185 that is currently in effect (or a pending programmed value), an amplitude safety margin 186, a minimum adapted amplitude 187, a pulse width 189, and a maximum test amplitude 190 used in a CM test. The ventricular channel 182 may include a programmable CSP safety margin 188 that may be applied by control circuit 80 for selecting the pacing pulse amplitude 185 that is currently in effect for the ventricular channel 182 (for CSP of the ventricles) as described below. It can be assumed that ventricular pacing is delivered as CSP according to the pacing pulse amplitude 185 and pulse width 189 programmed, by a user or automatically by IMD 14, for the ventricular channel 182. It is to be understood that when CSP is combined with LV myocardial pacing, e.g., when LV lead 47 is coupled to IMD 14 as shown in FIG. 3, a third LV channel may be included in the CM programming screen represented by diagram 180.

The CM mode 184 may be programmable individually for the atrial channel 181 and the ventricular channel 182 to be “off,” “monitoring” or “adaptive” in some examples. When programmed “off,” CM tests are not performed and CSP capture data is not acquired by IMD 14. When programmed to “monitoring,” IMD 14 may perform a CM test according to programmed control parameters to acquire cardiac electrical signals sensed during the CM test and/or data derived therefrom but may not make any automatic adjustments to the pacing output based on the results of the CM test. When programmed to “monitoring,” IMD 14 may record EGM signal(s) as the pacing pulse output is adjusted to store CM test EGM signal episodes that can be transmitted by IMD 14 for receipt by external device 50 or to network/cloud 75 for analysis and/or transmission to computing device 70 (see FIG. 1).

When programmed to “adaptive,” IMD 14 may perform CM tests according to programmed control parameters, store EGM signal(s) that are sensed during the CM test and adjust one or more control parameters based on an analysis of the EGM signal(s), such as increasing or decreasing a pacing pulse output (e.g., pacing pulse amplitude 185 and/or pulse width 189). When the capture management mode is programmed to “adaptive,” automatic adaptation of the pacing pulse output may be made by IMD 14 based on one or more capture threshold(s) determined from the EGM episode sensed during a CM test. For example, control circuit 80 of IMD 14 may determine a capture threshold, e.g., selective and/or or non-selective conduction system capture threshold, and adjust the pacing pulse amplitude to at least an amplitude safety margin 186 (which may be programmable) greater than determined capture threshold. In the example shown the safety margin is multiplicative in that it may be programmed to be 1, 1.5, 2.0, 2.5, or other multiple greater than the determined capture threshold. In other examples, an amplitude safety margin 186 may be additive in that it may be programmed to be a fixed offset greater than the determined capture threshold, e.g., 0.5, 1.0, 1.5, 2.0 or 2.5 volts (V) plus the determined capture threshold.

The pacing pulse amplitude 185 currently in effect can be displayed. In some examples, a user may reprogram the pacing pulse amplitude 185 by selecting either the atrial channel (181) or the ventricular channel (182) and entering a new pacing pulse amplitude. The amplitude safety margin 186 is the programmable safety margin that is added to or multiplied by a capture threshold determined during a CM test when the capture management mode is programmed to be “adaptive” as described above. The minimum adapted amplitude 187 may be programmable by a user and may be the minimum pacing pulse amplitude in volts that IMD control circuit 80 may automatically adjust the pacing pulse amplitude to when the capture management mode is programmed to be “adaptive.”

The CSP safety margin 188 can be optional and may be a programmable safety margin that may be applied by control circuit 80 to a capture threshold determined during CSP that captures at least a portion of the His-Purkinje conduction system. In some examples, the CSP safety margin 188 is an additional safety margin added to the capture threshold multiplied by (or added to) the amplitude safety margin 186 to set the pacing pulse amplitude of CSP pulses when the capture management mode 184 for the ventricular channel 182 is programmed to be “adaptive.” For example, if the conduction system capture threshold is determined to be 1.5 V and the safety margin is 2X, control circuit 80 may determine the CSP pulse amplitude to be 3 V (the capture threshold multiplied by the safety margin) plus the programmed CSP safety margin 188, e.g., plus 1 V or 4 V total in this illustrative example. The CSP safety margin 188 may be programmable between 0.25 and 3 V as examples. In other examples, the CSP safety margin 188 may be programmable as a multiplicative value instead of additive. For example, the CSP safety margin 188 may be programmable to be 1.0 to 2.0 times the conduction system capture threshold multiplied by the amplitude safety margin 186 (or plus an amplitude safety margin).

The CSP safety margin 188 may be automatically adjustable by control circuit 80 when “allow auto reprogram?” 191 is enabled. When automatic adjustment of the CSP safety margin 188 is enabled, the CSP safety margin may be increased or decreased when the conduction system capture threshold increases or decreases so that the CSP pulse amplitude is maintained at a desired safety margin relative to the capture threshold of a desired capture threshold type, e.g., non-selective or selective capture of a targeted CSP site.

A maximum test amplitude 190 may be programmable for each of the atrial channel 181 and the ventricular channel 182 in some examples. The maximum test amplitude 190 is the maximum pulse amplitude of the respective atrial pacing pulses or CSP pulses that are delivered by therapy delivery circuit 84 during an atrial or CSP CM test. The maximum test amplitude 190 may be programmable between 3 V and 10 V and may be programmable between 5 V and 8 V in some examples and may be set to a maximum available pacing pulse amplitude in some instances.

The pulse width 189 may be programmable for each atrial channel 181 and ventricular channel 182. The pulse width 189 may remain at the programmed value during all CM tests and during pacing of the respective atrial or ventricular chambers. It is recognized, however, that in other examples, the pacing pulse amplitude 185 could remain fixed during CM tests and during pacing of the respective atrial or ventricular chambers with the pacing pulse width 189 being adjusted during a CM test and automatically or manually reprogrammed based on capture threshold(s) determined from the CM test.

The CM programming screen represented by diagram 180 may include a capture management test schedule 192 that enables a user to program a CM test start time and repeat interval. For example, a user may program the CM test to be performed starting at 2:00 AM and repeat every 24 hours. The start time may be programmable to any time of day and the repeat interval may be programmable to be one hour, four hours, eight hours, 12 hours, 24 hours, 48 hours, 72 hours, or one week, as examples.

It is noted that the various values of programmable parameters listed for atrial channel 181 and the various values of programmable parameters listed for ventricular channel 182 are illustrative in nature and not to be considered limiting. A value for each programmable parameter may be selectable from a respective range of values by a user and/or automatically by control circuit 80.

Returning to FIG. 7, at block 154, control circuit 80 may determine that it is time for a CM test based on the programmed starting time and repeat interval, one or both of which may be user programmable as described above. In some examples, control circuit 80 may determine that it is time for a CM test based on other criteria than the programmed CM test schedule. For example, a CM test may be performed in response to a user command received via telemetry circuit 88.

Additionally or alternatively, control circuit 80 may determine it is time for a CM test in response to detecting a CM test trigger condition determined from a sensed cardiac electrical signal, such as a change in the QRS waveform morphology, a change in an activation time between a delivered CSP pulse and a QRS waveform feature, or another change in a QRS waveform feature such as a slope, amplitude, area, width, etc. that may be indicative of a change in capture type following a CSP pulse. A change in the QRS waveform morphology and/or one or more specific QRS waveform features may be detected by control circuit 80 by determining a QRS waveform feature that is compared to a value or a template value stored in memory 82 corresponding to a desired CSP capture type, which may be selective or non-selective conduction system capture, for example. In other examples, control circuit 80 may detect a change in a QRS waveform based on comparing a QRS waveform sensed following a most recent CSP pulse to a QRS waveform sensed following an earlier CSP pulse.

If control circuit 80 determines that it is not time for a CM test, control circuit 80 may wait at block 154 until it is determined to be time for a CM test. In some cases, IMD 14 may receive new CM control parameters at block 152 while waiting to perform the next CM test.

When control circuit 80 determines that it is time for CM test, control circuit 80 controls therapy delivery circuit 84 to deliver one or more CSP pulses at each of one or more CSP pulse outputs (e.g., one or more pacing pulse amplitudes using a fixed pulse width or vice versa) according to programmed CM control parameters at block 156.

During a CSP CM test, therapy delivery circuit 84 may begin the CM test by delivering N CSP pulses at the programmed maximum test amplitude (190 in FIG. 8). Therapy delivery circuit 84 may be controlled by control circuit 80 to deliver the CM test pacing pulses at a shortened pacing interval, e.g., at a shortened AV delay or at a shortened ventricular lower rate interval to overdrive pace any intrinsic ventricular electrical activity. Therapy delivery circuit 84 may be controlled by control circuit 80 to decrease the CSP pulse amplitude after every N delivered CSP pulses until a programmed minimum pulse amplitude is reached.

The minimum pulse amplitude used during the CM test may be the programmed minimum adapted amplitude 187 as shown in FIG. 8 or another programmed minimum pulse amplitude or the lowest available pacing pulse amplitude of therapy delivery circuit 84. In some examples, the programmable CM control parameters may include a programmable minimum test amplitude in addition to the minimum adapted amplitude.

The minimum test amplitude may be one decrement below the minimum adapted amplitude or other selectable setting. A user may select whether the CM test pacing pulses can be delivered below the programmed minimum adapted amplitude or not in some examples. In some instances, the pacing pulse amplitude may be decreased until loss of capture is detected by control circuit 80 from a sensed cardiac electrical signal (e.g., no evoked response following the CSP pulse), which may occur before reaching a minimum allowable pacing pulse test amplitude.

The number of CSP pulses delivered at each pacing pulse amplitude may be between one to twelve CSP pulses and may be three to five pacing pulses. In an illustrative example, during a CSP CM test, therapy delivery circuit 84 may deliver five CSP pulses at a maximum test amplitude of 5V and decrease the pulse amplitude by 0.25 V after every five CSP pulses using a fixed pulse width. The fixed pulse width may be between 0.1 and 2 ms as examples and may be 0.03 to 1.5 ms in some examples. In other examples, the pulse amplitude may be fixed at a programmed or default value, and the pulse width may be set to a maximum starting pulse width, e.g., 1.2 to 2.0 ms, that is decreased every N pacing pulses until a minimum pulse width, e.g., 0.03 to 0.5 ms, is reached.

During the CM test, sensing circuit 86 senses one or more EGM signals that may be passed to control circuit 80 for storing in memory 82. The EGM signal(s) recorded during the CSP CM test are expected to present one or more changes in QRS morphology as the CSP pulse output is decreased and the capture type changes and/or loss of capture occurs. A multi-channel EGM episode may be recorded during the CSP CM test. For example, at least one relatively near-field EGM signal and/or at least one relatively more far-field EGM signal may be stored in memory 82 as CSP CM test data. The near field EGM signal is a relatively local signal that may be recorded at or in the vicinity of the CSP site. The far field signal may be a relatively more global signal that may be recorded using at least one electrode positioned away from the CSP site and may be representative of the global coordination or synchrony of the ventricular electrical depolarization. Each of a near-field EGM signal and a far-field EGM signal may contain capture-related information useful in determining the type of CSP capture achieved at each pacing pulse output and for detecting changes in CSP capture that may warrant adjusting the CSP pulse output and/or alerting a clinician or other user, as further described below. In some examples, the atrial EGM signal, e.g., sensed using electrodes 20 and 22 shown in FIG. 1, may be stored with one or more ventricular EGM signals. Pacing event markers (e.g., CSP pulse markers and optionally atrial pacing pulse markers) and any intrinsic sensed P-wave markers and/or intrinsic sensed R-wave markers may be stored with the EGM episode.

In some examples, the EGM episode may be a single, continuous episode that encompasses the CM test starting from the first CSP test pulse to the last CSP test pulse. The EGM episode, which may be a multi-channel EGM episode including two or more EGM signals, may be recorded at a selected sampling rate (e.g., 128 to 512 Hz) over approximately 10 seconds, 30 seconds, 60 seconds, 120 seconds or other duration, depending on the time to complete the CM test. The time to complete the CM test can depend on the number of CSP pulses delivered at each pacing pulse output, number of test pacing pulse outputs used during the test, and the pacing rate. In other examples, the EGM episode may be recorded in a discontinuous manner, e.g., to reduce the memory capacity required to store the EGM episode and/or to enable a higher sampling rate of the EGM episode stored in memory 82. The EGM episode may be stored at block 158 as one or more QRS waveforms recorded for each CM test pacing pulse output. For instance, one to three QRS waveforms may be stored in memory 82 associated with each pacing pulse amplitude delivered during the CM test. Each QRS waveform may be recorded in memory 82 over a time interval that is, for example, 200 to 600 ms in duration or about 250 to 500 ms in duration, beginning from the delivered CSP pulse or following a post-pace blanking interval (e.g., 20 to 70 ms).

At block 160, telemetry circuit 88 may transmit the EGM signal episode stored in memory 82 during the CM test. The EGM signal episode is transmitted by IMD 14 for receipt by external device 50 or computing device 70 (which may be via relay device 45 and/or network/cloud 75), for example, for processing and analysis. As further described below, processing circuitry of the medical device system may analyze the EGM signal episode(s) for detecting alert conditions that may indicate a change in CSP capture that could warrant an adjustment or reprogramming of CSP pulse output. In the illustrative examples presented herein, the analysis of an EGM episode recorded during a CM test is described as being performed by processing circuitry after transmitting the recorded EGM episode from IMD 14, e.g., to external device 50 or to the network/cloud 75. External device processor 52, computing device processing circuitry 72, and/or cloud-based software of network/cloud 75 may perform the processing and analysis of the EGM episode for detecting changes in CSP capture. It is to be understood however, that the analysis of the EGM episode recorded by IMD 14 during a CM test may be performed in whole or in part (e.g., cooperatively) by any of the processing circuitry included in the medical device system, including IMD control circuit 80, external device processor 52, network/cloud 75, and/or computing device processing circuitry 72.

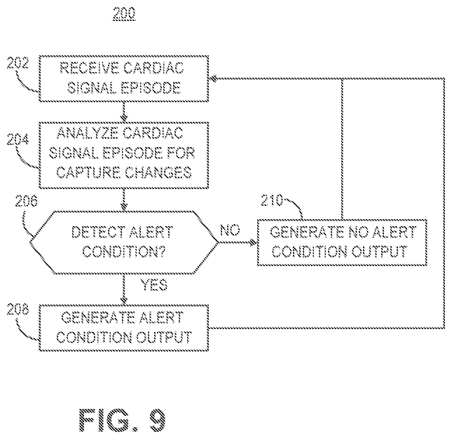

FIG. 9 is a flow chart 200 of a method that may be performed by processing circuitry of a medical device system, e.g., medical device system 10 of FIG. 1, for detecting an alert condition from an EGM episode recorded during a CM test according to some examples. As indicated above, the process of flow chart 200 may be performed by external device processor 52, computing device processing circuitry 72, cloud-based software executed by a processor or server of network/cloud 75 or any combination thereof.

At block 202, processing circuitry receives a transmitted cardiac signal episode obtained by an IMD configured to deliver CSP, e.g., IMD 14, during a CM test as generally described above in conjunction with FIG. 7. At block 204, the processing circuitry analyzes the cardiac signal episode for detecting capture changes. As described below, capture changes may be detected relative to a previous CM test. Capture changes may be detected when a different number or type of morphology changes occur during the CM test as CSP pulse output is decreased (or varied) and/or when morphology changes occur at different pacing pulse output levels than during a previous CM test. For the sake of convenience, the process of flow chart 200 and other flow charts and diagrams presented herein are described in conjunction with receiving a cardiac electrical signal episode as an EGM episode, where one or more EGM signals sensed by IMD 14 are transmitted for receipt by processing circuitry. However, it is to be understood that the cardiac signal episode may include one or more ECG signals (e.g., when cardiac electrical signal(s) is/are sensed using extra-cardiac subcutaneous, submuscular or substernal electrodes or external, surface or skin electrodes) in addition to or instead of the EGM signal(s).

In some examples, the changes in capture during a CM test may be detected without requiring identification or discrimination between capture types, such as discrimination between selective conduction system capture, non-selective conduction system capture and VMO capture. In other examples, processing circuitry of the medical device system may be configured to classify the capture type as one of multiple possible capture types, e.g., any of selective conduction system capture, non-selective conduction system capture, bilateral bundle branch capture, partial or complete right bundle branch capture, partial or complete left bundle branch capture, partial or complete His bundle capture, VMO capture, and/or loss of capture. When classifications of capture type are available, the processing circuitry may detect an alert condition when the number or types of capture detected are different compared to a previous CM test. When capture thresholds associated with each classification of capture type are available, e.g., the lowest pacing pulse output at which a particular capture type is identified, a change in a capture threshold associated with a capture type compared to a previous CM test may be detected as an alert condition at block 206.

In some examples, an alert condition may be detected at block 206 when a change in morphology or capture type occurs at a CM test pacing pulse output that is different than (e.g., greater than a threshold voltage difference from) the CM test pacing pulse output at which the corresponding morphology change or capture type change occurred during a previous CM test. In an illustrative example, when a particular change in QRS morphology or a change in capture type occurs at 3.0 V in the current CM test and the same QRS morphology change or change in capture type occurred at 3.25 V in a previous CM test, the 0.25 V change in CSP pulse output may be identified as an alert condition at block 206. In other examples, a relatively greater threshold change in pacing pulse output, e.g., at least 0.5 V, may be required to detect an alert condition. A relatively small change in pacing pulse output corresponding to the QRS morphology change indicating a change in capture type may be well within a safety margin of the pacing amplitude, for example.

When one or more changes in the CM test compared to a previous CM test are detected, the processing circuitry may detect an alert condition at block 206. If an alert condition is detected, the processing circuitry may generate an alert condition output at block 208. The alert condition output may include a message or notification of a CSP capture change that may be displayed by external device 50 or computing device 70. The alert condition output may include generating a display of a GUI including all or a portion of the EGM episode, which may include a marker of QRS morphologies that occur at different CSP pulse output levels compared to a previous CM test, QRS morphology that did not occur during a previous CM test, a QRS morphology that occurred in a previous CM test but did not occur in the present CM test, QRS morphology feature changes and corresponding CSP pulse output settings at which QRS morphology feature changes occur. In some examples, when the capture type is determined by the processing circuitry, changes in capture threshold (increase or decrease) of a particular capture type may be reported in the alert condition output generated at block 208.

In some cases, the processing circuitry determines that the EGM episode received for the most recent CM test is not different than the EGM episode determined for a previous CM test. For example, the analysis at block 204 may yield the same number of QRS morphology types or same number of classified capture types as a previous CM test. The analysis at block 204 may yield the same number of QRS morphology types or identified capture types each occurring at approximately the same pacing pulse outputs as a previous CM test. The “same” pacing pulse output may be defined to be within ±0.1 V, ±0.2 V, ±0.25 V, ±0.3 V, ±0.5 V or other specified threshold difference or as a percentage of the pacing output, a percentage of the amplitude safety margin, a percentage of the CSP safety margin or other specified threshold.

The processing circuitry may generate a notification that no alert condition is detected at block 210 when the EGM episode analysis does not meet criteria for detecting a change in the EGM episode compared to a previous CM test EGM episode. The EGM episode or portions thereof and/or data derived therefrom may be made available for display on external device 50 or computing device 70 for a clinician or other caregiver to review. However, because an alert condition was not detected, a notification that the CM test results and/or capture thresholds are stable or unchanged may be generated at block 210. After generating an output based on an alert condition detection or no alert condition detection, the processing circuitry may return to block 202 to wait for the next EGM episode transmission.

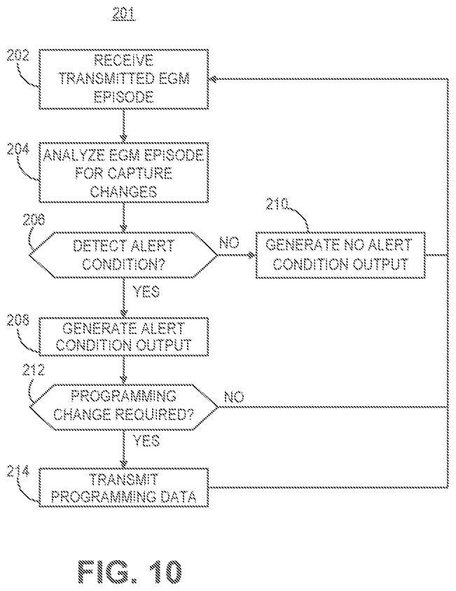

FIG. 10 is a flow chart 201 of a method that may be performed by processing circuitry of a medical device system, e.g., medical device system 10 of FIG. 1, for detecting and responding to a CSP capture change according to another example.

Identically numbered blocks in FIG. 10 correspond to like-numbered blocks in FIG. 9 described above. When processing circuitry detects an alert condition at block 206, one or more capture thresholds associated with CSP may have changed since a previous CM test. As a result, a programming change may be required to promote effective CSP at an output that is likely to achieve a desired capture type, e.g., selective or non-selective capture of at least a portion of the conduction system.

At block 212, the processing circuitry may receive a user input indicating that a programming change is required. A user that is informed by the alert condition output generated at block 208 may determine that a CSP control parameter, e.g., the pacing pulse amplitude and/or pulse width, may need to be increased or decreased due to a change in the CM test EGM episode detected by the processing circuitry relative to a previous CM test.

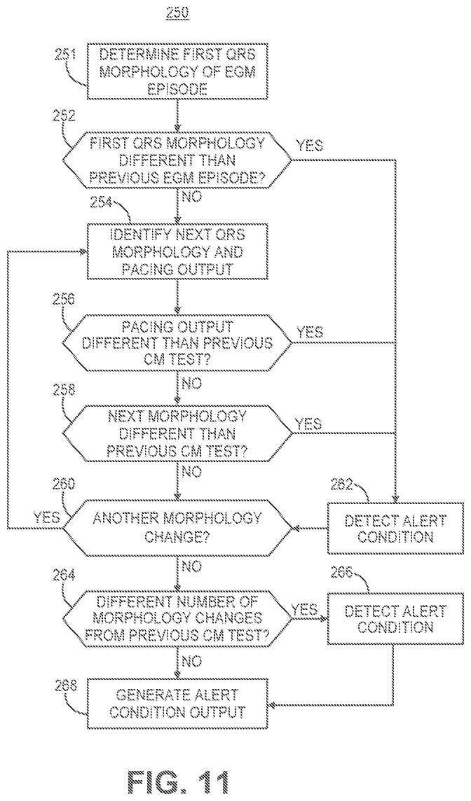

In other examples, the processing circuitry may be configured to determine that a programming change is required without necessarily requiring user input. For example, the processing circuitry may be configured to determine capture test data as further described below, e.g., in conjunction with FIG. 11. Capture test data may include identifying a QRS morphology type corresponding to each pacing pulse output (e.g., pulse amplitude) delivered during the CM test. The capture test data determined by the processing circuitry may additionally or alternatively include the pacing pulse output at which one QRS morphology type changes to another QRS morphology type (e.g., determine the capture threshold for each QRS morphology type) in the EGM episode. The number of different QRS morphology types may be identified without necessarily determining what types of capture are represented by the QRS morphology types. The capture test data determined by the processing circuity, however, may further include classifying the QRS morphology at each pacing pulse output according to one of multiple capture types. The processing circuitry may determine the capture threshold of each classified capture type. The number of QRS morphology types and/or number of classified capture types of the EGM episode may be determined as capture test data by the processing circuitry in the analysis performed at block 204.

When the capture test data determined by the processing circuitry changes compared to a previous CM test, the processing circuitry may determine that a programming change is required at block 212. A change in the capture test data may be detected as a different number of QRS morphology types, different number of capture type classifications, or a different pacing pulse output (or range of pacing pulse outputs) at which a QRS morphology type or classified capture type or loss of capture occurs compared to a previous CM test. In response to detecting the change in the capture test data, the processing circuitry may determine that a programming change is required at block 212.

In examples that processing circuitry is configured to determine one or more capture thresholds, the processing circuitry may determine that a programming change is required based on a threshold difference in a capture threshold compared to a previously determined capture threshold of an earlier CM test. In other examples, the processing circuitry may determine that a programming change is required when a specific morphology feature or QRS morphology type is determined to be different at the currently programmed CSP pulse output (e.g., the current pacing pulse amplitude that is in effect since the last CM test) or any of the test pacing pulse outputs during the CM test compared to the QRS morphology type at the same pacing pulse output in a previous CM test.

When the processing circuitry determines that a programming change is required based on received user input or analysis of the EGM episode, the medical device system may receive programming input from a user, e.g., via user display/interface 78 of computing device 70 or via user interface 56 and/or display unit 54 of external device 54. The processing circuitry may control the communication circuit 76 of computing device 70 or telemetry unit 58 of external device 50 to transmit programming commands via an associated communications network or wireless telemetry link at block 214. IMD 14 may receive the programming commands and adjust one or more CSP control parameters accordingly.

For example, the pacing pulse output, e.g., CSP pulse amplitude or pulse width, may be increased if an indication of increased capture threshold of a desired capture type is determined from the EGM episode to promote effective CSP. In other instances, the pacing output may be decreased if an indication of decreased capture threshold is determined in order to conserve IMD power source 98. In some examples, programming commands may include changes to the amplitude safety margin, the CSP safety margin, the maximum test amplitude, the minimum adapted amplitude and/or the CM test schedule start time and/or repeat interval. In some examples, the programming commands may include a change in the capture management mode, e.g., between an adaptive mode, monitoring mode or off. Example programmable parameters that may be programmed based on CM test EGM episode analysis are described above in conjunction with FIG. 8. If no programming change is required (“no” branch of block 212) or after transmitting programming data (e.g., commands and settings) at block 214, the processing circuitry may return to block 202 to wait for the next transmitted EGM episode.

FIG. 11 is a flow chart 250 of a method for detecting an alert condition from an EGM episode by processing circuitry of a medical device system according to some examples. The process of flow chart 200 may be incorporated in the methods of flow charts 200 or 201, in whole or in part, for detecting an alert condition by identifying a change in an EGM episode received for a most recent CM test compared to an EGM episode received for a previous CM test.

At block 251, the processing circuitry may determine one or more features of the QRS waveform morphology following the CM test pacing pulse(s) delivered at the first pacing pulse output of the CM test, e.g., at the maximum CM test pacing pulse amplitude. A first QRS morphology of the EGM episode may be characterized by determining one or more features such as any of a peak amplitude, peak-to-peak amplitude, QRS width, QRS area, activation time from the delivered pacing pulse to a fiducial point (e.g., maximum peak amplitude) of the QRS waveform, a maximum positive slope, a maximum positive slope time (from the delivered pacing pulse), a maximum negative slope, a maximum negative slope time, a morphology matching score or other QRS morphology feature or any combination thereof. The morphology matching score may be determined by wavelet transform, correlation analysis or other techniques to determine a matching score between the QRS waveform and a stored QRS template, which may correspond to a known capture type, for example.

At block 252, the processing circuitry may compare the first QRS morphology of the EGM episode to the first QRS morphology of a previous CM test EGM episode. In some instances, the first QRS morphology of the EGM episode may be compared to a QRS morphology that is not the first QRS morphology of the previous CM test if the starting maximum pacing pulse output has changed. In this case, the first QRS morphology of the current EGM episode corresponding to the starting (e.g., maximum) CM test pacing pulse output may be compared to the QRS morphology identified for the same pacing pulse output in the previous CM test, which may or may not be the first, starting pacing pulse output. An EGM episode recorded for at least one previous CM test and or capture test data determined therefrom for the given patient may be stored in external device memory 53, computing device memory 74, or on a server of network/cloud 75. In this way, processing circuitry of the medical device system can make comparisons between the current EGM episode and previous CM test EGM episode(s). Additionally or alternatively, QRS morphology data (such as any of the QRS features listed above) and the associated pacing pulse output determined from at least one previous EGM episode may be stored in memory of the medical device system.

If one or more features of the first QRS morphology present in the EGM episode are different than the corresponding feature(s) of the QRS morphology present in a previous EGM episode (from a previous CM test) for the same pacing pulse output, the processing circuitry may detect an alert condition at block 262 (“yes” branch of block 252). Assuming that both the current and the previous CM test started at the same maximum test amplitude, for example, the QRS morphology is expected to be the same in both EGM episodes if a change in capture threshold has not occurred. If the first QRS morphology present in the current EGM episode is determined to be different that the first QRS morphology present in a previous EGM episode, a change in CSP capture may have occurred. In examples that include different starting test pacing pulse outputs, e.g., different maximum test amplitudes, the processing circuitry may determine and compare QRS morphology features corresponding to the earliest, equal pacing pulse outputs delivered during the current CM test and a previous CM test.

If the first QRS morphology of the EGM episode is not different than the first QRS morphology (or QRS morphology of the equivalent pacing pulse output) of a previous CM test EGM episode, the processing circuitry may advance to block 254 to identify a next QRS morphology, different than the first QRS morphology, that occurs in the EGM episode. When the processing circuitry does detect an alert condition (block 262) based on a different first QRS morphology (“yes” branch of block 252), the processing circuitry may determine if the EGM episode includes another QRS morphology (other than the first QRS morphology) at block 260. If so, the processing circuitry may proceed to block 254 to identify the next QRS morphology features associated and the associated pacing pulse output.

When a second QRS morphology is identified in the EGM episode, e.g., after one or more decrements in the pacing pulse amplitude during the CM test, the processing circuitry may determine if the second QRS morphology and associated pacing pulse output at which the second QRS morphology first appears in the EGM episode represent a change from a previous CM test EGM episode. In some instances, the second QRS morphology that occurs in the EGM episode as the pacing pulse output is decreased may be the same QRS morphology as a second QRS morphology in a previous CM test EGM episode. The second QRS morphology of the current EGM episode may be determined to be the same as the second QRS morphology based on a comparison of waveform morphology features or using morphology matching techniques. However, the highest pacing pulse output at which the second QRS morphology first appears in the current EGM episode may be different than the highest pacing pulse output at which the second, matching QRS morphology first appears in the previous CM test EGM episode. When the pacing output at which the second QRS morphology appears in the current EGM episode is different than the pacing output at which the second QRS morphology appears in a previous CM test EGM episode (“yes” branch of block 256), the processing circuitry may detect an alert condition at block 262.

In other instances, the second QRS morphology in the current EGM episode may be different than the second QRS morphology that appears in the previous CM test EGM episode. When the second QRS morphology of the current EGM episode is different than the second QRS morphology of the previous CM test EGM episode (“yes” branch of block 258), the processing circuitry may detect an alert condition at block 262.

Accordingly, a CSP capture change may be detected by the processing circuitry when a QRS morphology in the current EGM episode is different than the QRS morphology that occurs at the same pacing pulse output in a previous CM test EGM episode. Additionally or alternatively, a CSP capture change may be detected by processing circuitry when the same QRS morphology first appears at a different pacing pulse output in the current EGM episode compared to a previous EGM episode. A CSP capture change may be detected when the nth QRS morphology identified during the CM test, e.g., as the pacing output is decreased, is different than the nth QRS morphology identified in a previous CM test. When a CSP capture change is detected based on comparisons between a current EGM episode and a previous CM test EGM episode, the processing circuitry may detect an alert condition at block 262.

The process of identifying a next QRS morphology that appears in the EGM episode as the pacing output is decreased (or increased or otherwise varied) may continue for identifying one or more CSP capture changes that may occur at one or more pacing pulse outputs in the current CM test EGM episode compared to a previous CM test EGM episode. When no further morphology changes are identified within the current EGM episode (“no” branch of block 260) or when the last QRS waveform of the EGM episode has been evaluated, the processing circuitry may determine at block 264 if the total number of QRS morphology types presented in the current EGM episode is different than the total number of QRS morphology types presented in the previous CM test EGM episode.

In some instances, non-selective capture of the conduction system with ventricular myocardial capture may occur until the pacing pulse output is decreased below a conduction system capture threshold. Below the conduction system capture threshold, VMO capture may occur until the pacing pulse output is decreased below the VMO capture threshold, at which point loss of capture may occur. In this illustrative example, three distinct QRS morphologies may be present in the CM test EGM episode corresponding to non-selective conduction system capture, VMO capture, and loss of capture. At other times, in the same patient, selective conduction system capture, non-selective conduction system capture with ventricular myocardial capture, VMO capture and loss of capture may occur during the CM test. In this case, four distinct QRS morphologies may be present in the EGM episode corresponding to each type of capture. In still other examples, different QRS morphologies identified from an EGM episode sensed during a CM test may correspond to any of (with no limitation intended): selective partial His bundle capture, selective complete His bundle capture, non-selective partial His bundle capture, non-selective complete His bundle capture, selective LBB capture, non-selective LBB capture, selective bilateral BB capture, non-selective bilateral BB capture, selective RBB capture, non-selective RBB capture, VMO capture, and loss of capture.

As such, the processing circuitry may determine if a different number of QRS morphologies (or different number of QRS morphology changes) occur in the EGM episode at block 264 compared to a previous CM test. When a different number of QRS morphologies are identified, the processing circuity may detect an alert condition at block 266. When the number of QRS morphologies identified in the EGM episode has not changed compared to a previous CM test, the processing circuitry may advance directly to block 268.

At block 268, the processing circuitry may generate an alert condition output based on the number and type of alert conditions that are detected (which in some instances may be no alert conditions detected). The generated alert condition output may be received by and stored in memory of the medical device system, The alert condition output may be a notification to the clinician or other caregiver indicating whether or not an alert condition is detected from the current EGM episode. The alert condition output may include data presented in a display, e.g., in a GUI, or a report, e.g., a summary table, graph etc., of CSP capture changes identified as alert conditions, which may include one or more changes in identified QRS morphologies, a change in the pacing output at which a given QRS morphology occurs, and/or a change in the number of QRS morphologies identified. The alert condition output may include a display of the EGM episode or snippets from the EGM episode representative of any identified CSP capture changes detected as alert conditions.

In some examples, the generated alert condition output may include a recommended or automatic programming change. For example, when the lowest pacing pulse output at which a given QRS morphology occurs at is increased compared to a previous CM test, an automatic or recommended increase in the pacing pulse amplitude, pulse width, amplitude safety margin or CSP safety margin may be provided as output at block 268. When the lowest pacing pulse output at which a given QRS morphology occurs is decreased, an automatic or recommended decrease in the pacing pulse amplitude, pulse width, amplitude safety margin or CSP safety margin may be provided as output at block 268.

In other examples, if no change in CSP capture is identified as an alert condition for a threshold number of CM tests, the repeat interval of performing CM tests may be increased. If a change in CSP capture is identified as an alert condition, the repeat interval for performing CM tests may be decreased so that CM tests can be performed more often to enable programming changes to CSP pulse output as needed. Adjustments to the maximum CM test pulse output and/or minimum CM test pulse output may be made or recommended by the medical device processing circuitry based on detected alert conditions.

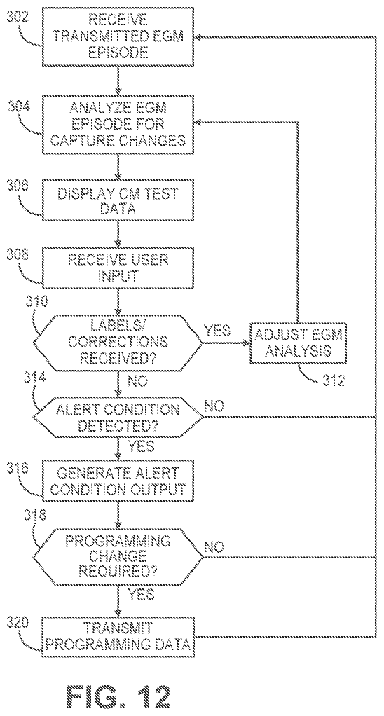

FIG. 12 is a flow chart 300 of a method for analyzing CM test EGM episodes for detecting changes in CSP capture by processing circuitry of a medical device system according to another example. At block 302, a CM test EGM episode is received by processing circuitry of the medical device system. At block 304, the processing circuitry analyzes the EGM episode for detecting morphology changes within the EGM episode indicative of changes in capture type that occur during the CM test.

The processing circuitry may distinguish between capture type based on an analysis of the QRS morphology following each CM test pacing pulse. Different capture types that may be identified by the processing circuitry are listed as examples above. The processing circuitry may determine QRS waveform features following CM test pacing pulses. For instance, QRS waveform feature(s) may be determined from one or more EGM signals sensed during a post-pace window following a CSP pulse delivered during the CM test. The QRS waveform features can be determined for comparison to each other and/or various thresholds, ranges or other criteria in a capture detection and classification algorithm executed by the processing circuitry. Some examples of QRS waveform features that may be analyzed for classifying different capture types during CSP are generally disclosed in U.S. Application Publication No. 2020/0406041 (Cao, et al.), U.S. patent application Ser. No. 17/735,628 (Zhou, et al), and U.S. patent application Ser. No. 17/370,303 (Cao, et al.), the content of all of which is incorporated herein by reference in its entirety.

In other examples, the processing circuitry may provide the EGM episode or selected portions thereof as input to a machine learning model or other AI model for analyzing the input and providing a capture type output, which may include a capture type classification and an indication of the classification confidence level (e.g., as a percentage confidence). AI techniques used for CSP capture type classification may include deep learning techniques such as convolutional neural networks (CNN), residual CNN, feed-forward neural network (FFNN), recurrent neural network (RNN), transformer, or other machine learning techniques such as decision tree, random forest model, or other machine learning approaches for establishing a model for classifying a delivered CSP pulse (and corresponding post-pace QRS waveform) according to capture type. Each CSP pulse (or each CSP pulse output) delivered during the CM test may be classified according to capture type based on the post-pace, unknown cardiac signal input from the received EGM episode.

A CSP capture classification model implemented in the processing circuitry of the medical device system may learn from cardiac electrical signal data obtained from a population of patients using machine learning. In some examples, the processing circuitry is trained to classify a post-pace EGM waveform in the CM test EGM episode input received from IMD 14 by performing the capture classification algorithm using at least one other template beat input representative of a QRS waveform following a CSP pulse having a pre-selected pacing pulse output. The template beat input can be a patient specific template generated from one or more QRS waveforms acquired following CSP pulses delivered at a specified pacing pulse amplitude.

As described above, an EGM episode may include one or more cardiac electrical signals. For example, two EGM signals including a near field EGM signal and a far field EGM signal may be included in the EGM episode. The near field signal may be a bipolar ventricular EGM signal, an EGM signal sensed with a relatively small inter-electrode distance and/or an EGM signal sensed using an electrode near the CSP site to generally provide a signal representative of relatively localized ventricular electrical activity. A far field EGM signal may be a unipolar ventricular EGM signal, an EGM signal sensed with a relatively larger inter-electrode distance and/or sensed using an electrode located away from the CSP site to generally provide a signal more representative of global ventricular electrical activity than the near field EGM signal. One or both of a near-field EGM signal and/or far-field EGM signal may be provided as input to an AI model as multi-channel input. In other examples, three, four or more cardiac electrical signals may be available for input to the AI model for capture type classification. Other input signals that the AI model may be trained on and may subsequently receive for generating a capture classification of an unknown post-pace signal in the CM test EGM episode may include the CM test pacing pulse amplitude (and/or width), a derivative of a cardiac electrical signal waveform, one or more QRS waveform templates, and/or one or more QRS waveform morphology features determined from the EGM episode, e.g., an activation time, signal width, signal area, peak amplitude, peak-to-peak amplitude, peak polarity, etc. Examples of AI techniques that may be implemented in conjunction with the techniques disclosed herein to provide a capture type classification of CM test pulses delivered during the recorded EGM episode are generally disclosed in U.S. Patent Application No. 63/337,769 (Berlin, et al.), the entire content of which is incorporated herein by reference.

In addition to analyzing the EGM episode for morphology changes that occur within the EGM episode as CM test pacing pulse output is changing, the processing circuitry may analyze the EGM episode for identifying capture-related changes compared to a previous CM test EGM episode. A previous CM test EGM episode received from IMD 14 and/or capture test data derived therefrom may be stored in memory of the medical device system for comparison to a subsequent CM test EGM episode. Capture-related changes relative to a previous CM test EGM episode may include the number of QRS morphologies present in the current EGM episode compared to a previous CM test EGM episode, difference(s) in the CM test pacing pulse output(s) at which QRS morphology change(s) occur compared to a previous CM test EGM episode, and/or differences in the QRS morphology at the same pacing pulse output compared to a previous CM test EGM episode, as examples. The capture-related changes compared to a previous CM test EGM episode may be identified as alert conditions at block 304, e.g., according to any of the examples described herein.

At block 306, the processing circuitry may generate CM test data for display to a clinician or other user. The CM test data may include the EGM episode or portion(s) thereof, CM test pacing pulse output(s), CSP pulse markers, atrial pacing pulse markers, intrinsic sensed event markers, changes in QRS morphology features that occur within the EGM episode as the CM test pacing pulse output is changed and/or capture type classifications (if available). The CM test data may additionally or alternatively include capture-related changes identified between the current EGM episode and a previous CM test EGM episode as described above. The CM test data may include any alert conditions detected by the processing circuitry based on detected differences between the current EGM episode and a previous CM test EGM episode according to any of the examples described above. The CM test data may be displayed on external device display unit 54 and/or computing device user display/interface 78, e.g., in a GUI.

At block 308, the processing circuitry may receive user input identifying one or more EGM episode waveforms (and associated CM test pacing pulses) as a capture type or a significant morphology change. In some examples, QRS waveforms of the EGM episode at different CM test pacing pulse outputs may be displayed for a clinician or other user to enable the user to label one or more QRS waveforms according to capture type or according to a QRS morphology type, e.g., morphology 1, morphology 2, etc. In some examples, QRS waveforms of the EGM episode may be classified according to the capture type by the processing circuitry. A clinician or other user may re-label a capture type that may be mis-classified by the processing circuitry based on expert truthing. In some instances, a user input received at block 308 may be a truth input confirming a capture type classification or QRS waveform morphology labeled in the GUI according to determination made by the processing circuitry. In various examples, the user input received at block 308 may include labels or annotations of one or more paced beats in the EGM episode, which may be corrections or confirmations of labels or annotations generated by the processing circuitry or may be labels or annotations applied to unlabeled paced beats of the EGM episode. In some cases, a user may label or annotate one or more paced beats of the EGM episode at each of the CM test pacing pulse outputs. In other examples, a user may label each morphology type (which may occur at multiple CM test pacing pulse outputs) as a numbered or otherwise classified morphology type or as a specifically classified capture type.

If user input is not received at block 310, or if no new labels or corrected labels are received such than any user input that is received at block 308 is to confirm existing labels or annotations generated by the processing circuitry (“no” branch of block 310), the processing circuitry may advance to block 314. When user input labels are received at block 310 that includes new labels and/or corrections to labels or annotations generated by the processing circuitry, the processing circuitry may return to block 304 to re-analyze the EGM episode for capture-related changes. In this way, expert truthing of labeled QRS morphologies that occur during the CM test can be used as feedback in the algorithms executed by the medical device system processing circuitry for detecting alert conditions of the CM test EGM episode. The user input may be used by the processing circuitry to personalize the algorithm to a specific patient and/or make improvements to an algorithm used for a population of patients, e.g., in a cloud-based computing algorithm or AI model. As such, in some examples at block 312 the processing circuitry may adjust the algorithm or criteria (such as morphology feature thresholds, ranges or other criteria) based on the user input received at block 310 to improve detection of QRS morphology changes within the EGM episode of the current CM test and/or to improve detection of capture-related changes compared to a previous and/or future CM test EGM episode. When an AI model is used for classifying or labeling QRS morphologies, the user input may be used for re-training the model to improve confidence levels of labels or capture type classifications output by the model.

The EGM episode analysis may be repeated at block 304 one or more times based on the user input received at block 308. In other examples, after receiving the user input at block 308, the processing circuitry may update or adjust the EGM analysis algorithm at block 312 without repeating the analysis of the current EGM episode. Rather, after making any adjustments at block 312 to the algorithm or criteria for detecting QRS morphology changes within the EGM episode and/or for detecting capture-related changes between the current EGM episode and a previous and/or future CM test EGM episode, the process of flow chart 300 may advance to block 314. In some examples, the adjustments to the EGM analysis algorithm or criteria made at block 312 may be omitted. The processing circuitry may rely on the EGM analysis algorithm and criteria for detecting QRS morphology changes and/or making capture type classifications without making adjustments at block 312, but the processing circuitry may use the user input received at block 308 for detecting an alert condition at block 314.

At block 314, the processing circuitry may detect one or more alert conditions indicating a capture-related change relative to a previous CM test EGM episode. The processing circuitry may detect the alert condition(s) based on the first EGM episode analysis performed at block 304 and any user input received at block 308 (without necessarily adjusting or repeating the EGM episode analysis after receiving user input). In other examples, the processing circuitry may detect the alert condition(s) based on an EGM episode analysis repeated at block 304 after receiving the user input at block 308 (and optionally making any adjustments to the EGM episode analysis at block 312). Any of the example alert conditions described above may be detected at block 314 based on a comparative analysis between the current EGM episode after receiving any user input for expert truthing of QRS morphology types and/or capture classifications and a previous CM test EGM episode (which may also include user-entered labels of QRS morphology types and/or capture classifications). In some instances, the processing circuitry may not detect an alert condition when the EGM episode does not differ significantly from a previous CM test EGM episode. In this case, it is to be understood than an appropriate output of no capture-related changes detected, no alert condition detected and/or no programming change needed may be generated and displayed by the medical device system. The process may return to block 302 to wait to receive the next EGM episode transmitted by IMD 14 when no alert condition is detected at block 314.

When an alert condition is detected at block 314, an alert condition output may be generated at block 316. For example, a display of any detected capture-related changes relative to a previous CM test EGM episode may be generated with the detected differences in the current EGM episode compared to the previous EGM episode being conspicuously presented or annotated in a GUI. Examples of alert condition outputs that may be generated at block 316 are described above. In some examples, a recommended programming change, e.g., an increased or adapted pacing pulse output, amplitude safety margin, CSP safety margin, minimum adjusted amplitude, maximum test amplitude, minimum test amplitude, and/or CM test schedule changes may be displayed at block 316.

In other examples, the processing circuitry may receive a user input at block 318 indicating a programming change. A clinician or other user may select a programming change, e.g., to any of the example programmable parameters listed above and described in conjunction with FIG. 8, based on the displayed CM test data and/or the generated alert condition output. If a programming change is not required, the process may return to block 302 to wait to receive the next transmitted CM test EGM episode.

When a programming change is required as determined at block 318 by the processing circuitry based on a detected alert condition or based on receiving a programming command from a user, the corresponding programming data is transmitted to the IMD 14 at block 320. Computing device 70 may be used by a clinician or other user to remotely accept and transmit programming commands via the network/cloud 75 and optionally a relay device 45 as shown in FIG. 1. In other examples, external device telemetry unit 58 may transmit programming data to IMD 14. The processing circuitry of the medical device system may be configured to automatically select a programming change to one or more CM control parameters. The corresponding programming commands may be transmitted without requiring clinician or other caregiver approval. In other cases, a programming command may be transmitted after a clinician or other user accepts and authorizes the programming command. In still other cases, the clinician or other user enters the programming command(s) and initiates the transmission of the programing command(s) to IMD 14. After transmitting any programming data needed when a programming change is required (or recommended), the processing circuitry may return to block 302 to wait to receive the next CM test EGM episode transmitted by IMD 14.

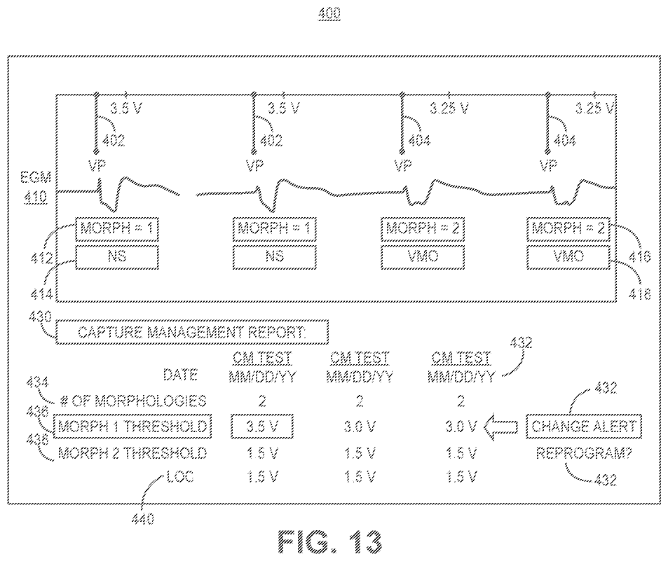

FIG. 13 is a diagram 400 of a GUI that may be generated by medical device processing circuitry for displaying capture test data to a user according to some examples. In the example shown, CSP pulse markers are shown, labeled as ventricular pace (VP) markers, 402 and 404. The CSP pulse markers 402 and 404 may be labeled with the corresponding pacing pulse amplitude as shown (in this example 3.5 V or 3.25 V). In other examples, each of the CSP pulse markers 402 and 404 may be labeled with the pulse width, an AV delay or ventricular lower rate interval, the pacing mode (e.g., DDD, VVI, etc.) and/or other CSP control parameter(s).

The GUI of diagram 400 is further shown to include a portion of the EGM episode 410 received for the most recent CM test. The QRS waveforms following each CSP pulse marker 402 and 404 in the EGM episode 410 can be observed by a user. While only a portion of the EGM episode 410 is shown as being displayed in FIG. 13, it is to be understood that the entire EGM episode may be displayable in the GUI with the ability for a user to scroll back and forth and/or zoom in and out on segments of the EGM episode. In some examples, snippets of the EGM episode 410 may be selected by the processing circuitry for display in a GUI for displaying representative QRS waveforms at each CM test pacing pulse output and/or representative QRS waveforms when a change in the QRS morphology occurs from one paced beat to the next.

One or more QRS waveforms of the EGM episode 410 may be labeled, e.g., according to a morphology type 412 (shown numbered as morphology 1, 2, etc.) and/or according to a capture type classification 414. The processing circuitry may analyze the EGM episode to identify the number of distinct QRS waveform morphologies that occur during the CM test. In the example shown, two distinct morphology types, shown labeled morphology 1 and morphology 2, are identified before LOC is detected. The intrinsic QRS morphology when loss of capture occurs (or loss of a QRS waveform if complete AV block is present) may be counted as a morphology type in some examples. In other examples, the EGM signal waveform following a CSP pulse that results in LOC may not be counted as a morphology type by the processing circuitry because it is not associated with capture of the cardiac tissue and the QRS waveform may be absent. Morphology type labels may be generated by the processing circuitry to annotate the QRS waveforms of the EGM episode, e.g., as type 1, 2, 3, etc., or as type A, B, C etc. or other labeling system, to distinguish the distinct QRS waveform morphologies that the processing circuitry identifies as being different morphologies present during the CM test.

When the processing circuitry is configured to classify the identified QRS waveform morphologies according to a capture type, a capture type classification label 414 may be generated for annotating one or more QRS waveforms in the displayed portion of the EGM episode 410. In the example shown, the first two CSP pulses marked by VP markers 402 are shown delivered with a 3.5 V pacing pulse amplitude. The next two CSP pulses marked by VP markers 404 are shown delivered with a 3.25 V pacing pulse amplitude. A change in morphology from morphology type 1 to morphology type 2 occurs when the CSP pulse amplitude is decreased from 3.5 V to 3.25 V. In this illustrative example, the processing circuitry may identify morphology type 1 as being non-selective (NS) capture of at least a portion of the His-Purkinje system that occurs with capture of the ventricular myocardium. When the CSP pulse amplitude is decreased to 3.25 V, the morphology type 2 is identified by the processing circuitry as being VMO capture. In this instance, conduction system capture is lost at 3.25 V, indicating a NS conduction system capture threshold of 3.5 V. The processing circuitry may generate a NS capture type label 414 to annotate the first two QRS waveforms associated with VP markers 402 and a VMO capture type label 418 to annotate the second two QRS waveforms associated with VP markers 404. As the pacing pulse output is further decreased, a LOC label may be generated when the morphology type 2 changes (or disappears).

In some examples, a user interacting with the GUI represented by diagram 400 may select any of the morphology type labels 412 and 416 and/or capture type labels 414 and 418 that are displayed in the GUI and enter a corrected label. In other examples, the processing circuitry may identify and label distinct morphology types without labeling the capture types. A user may label the capture types and may, in the process of labeling the capture types, enter a corrected label of a QRS morphology type. As described above, this user input may be used by the processing circuitry to re-evaluate an EGM episode for identifying changes in an EGM episode relative to a previous CM test EGM episode. In some examples, the user input labels of one or more QRS morphologies according to morphology type and/or capture type classification may be used by the processing circuitry to adjust the algorithm or criteria being used to identify QRS morphologies and/or classify capture types. For example, a new threshold, range or other criteria used for identifying different QRS morphology types or capture types may be established based on the re-labeled morphology waveform or one or more specific features of the morphology waveform.

The GUI may include a capture management report (CM report) 430 that may present summary data derived from the EGM episode by the processing circuitry. The CM report 430 may include data relating to the current CM test and optionally one or more previous CM tests. In the example shown, data determined from three CM tests are shown, each with a corresponding date 432. It is recognized that if more than one CM test is performed per day according to the programmed CM test repeat interval, the date and time of each CM test may be displayed. In various examples, the processing circuitry may generate CM test data that can include the number of morphology types 434 that are identified in the respective CM test EGM episode, the capture threshold 436 and 438 of each morphology type (e.g., expressed as the pulse amplitude in volts as shown or as the pulse width in milliseconds), and/or the CSP pulse output at which LOC 440 occurs. In this way, changes in the number of morphology types and/or the capture threshold associated with each morphology type may be displayed for comparison between CM tests. In some examples, a representative QRS waveform of each morphology type may be displayed for each CM test. In this way, a change in QRS morphologies relative to each other may be observed.

As described in various examples given above, the processing circuitry may be configured to identify one or more changes in the EGM episode for the current CM test compared to a previous CM test EGM episode as an alert condition. In in the illustrative example of FIG. 13, the capture threshold for morphology type 1 increased from 3 V (in two previous CM tests) to 3.5 V in the current CM test. This change in capture threshold associated with a morphology type may be detected as an alert condition by the processing circuitry. The processing circuitry may generate an alert condition output which may include conspicuously displaying data relating to the CM test change identified as an alert condition. As shown in FIG. 13, the increased capture threshold associated with morphology type 1 may be highlighted and/or labeled as a change alert 432 to notify the clinician or other user of the detected alert condition. The GUI may have a reprogram button 432 that a user may select for opening a programming window for reprogramming one or more CM control parameters as described above, e.g., the programming GUI shown in diagram 180 of FIG. 8. One or more alert conditions may be identified and conspicuously displayed in the capture management report 430 to notify a clinician that reprogramming may be warranted.

It should be understood that, depending on the example, certain acts or events of any of the methods described herein can be performed in a different sequence, in parallel, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single processor, circuit or unit for purposes of clarity, it should be understood that the techniques of this disclosure may be performed by a combination of processors, units or circuits associated with, for example, a medical device system.

In one or more examples, the functions described may be implemented in hardware, software, firmware, or any combination thereof. If implemented in software, the functions may be stored as one or more instructions or code on a computer-readable medium and executed by one or more hardware-based processing units. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device system has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device system, comprising:
processing circuitry configured to:
receive a cardiac electrical signal episode sensed during a conduction system capture management test;
determine capture test data from the cardiac electrical signal episode;
compare the capture test data to previous capture test data determined from a previous conduction system capture management test;
generate an output based on the comparing of the capture test data to the previous capture test data; and
detect an alert condition corresponding to a change in the capture test data compared to the previous capture test data;
a memory configured to store the output; and
a display unit in communication with the processing circuitry for receiving the generated output and displaying data in a user interface based on the generated output and the alert condition.

2. (canceled)

3. The medical device system of claim 1 wherein the processing circuitry is configured to:
determine the capture test data by identifying a number of different QRS waveform morphologies in the cardiac electrical signal episode; and
detect the alert condition by determining that the number of different QRS waveform morphologies in the cardiac electrical signal episode is different than a previous number of QRS waveform morphologies identified in a previous cardiac electrical signal episode sensed during the previous conduction system capture management test.

4. (canceled)

5. The medical device system of claim 3 wherein the processing circuitry is further configured to determine the capture test data by determining a capture threshold for one or more of the different QRS morphologies identified in the cardiac electrical signal episode.

6. The medical device system of claim 5, wherein the processing circuitry is further configured to detect an alert condition by determining that a capture threshold determined for at least one of the QRS morphologies identified in the cardiac electrical signal episode is different than a previously determined capture threshold determined for the respective QRS morphology identified in the previous cardiac electrical signal episode sensed during the previous capture management test.

7. The medical device system of claim 1 wherein the processing circuitry is further configured to determine the capture test data by classifying at least one QRS waveform in the cardiac electrical signal episode according to a capture type.

8. The medical device system of claim 7 wherein the processing circuitry is further configured to:
determine the capture test data by classifying a plurality of QRS waveforms in the cardiac electrical signal episode according to a plurality of capture types; and
determine a capture threshold of one or more of the classified capture types of the plurality of capture types of the cardiac electrical signal episode.

9. (canceled)

10. The medical device system of claim 8 wherein the processing circuitry is further configured to detect the alert condition by determining a change in the capture threshold determined for at least one of the classified capture types compared to a previous capture threshold determined for the at least one of the classified capture types in the previous conduction system capture management test.

11. The medical device of claim 8 wherein the processing circuitry is further configured to determine an alert condition by determining that a classified capture type of the plurality of capture types occurs at a pacing pulse output of a plurality of pacing pulse outputs of the conduction system capture management test and that the classified capture type of the plurality of capture types does not occur at the pacing pulse output of the plurality of pacing pulse outputs of the previous conduction system capture management test.

12. The medical device system of claim 1 wherein the processing circuitry is further configured to receive, via the user interface, a user input labeling at least one QRS waveform of the cardiac electrical signal episode according to at least one of a morphology type or a capture type.

13. The medical device system of claim 12, wherein the processing circuitry is further configured to adjust the determination of capture test data from the cardiac electrical signal episode in response to receiving the user input.

14. The medical device system of claim 1 wherein the processing circuitry is further configured to determine the capture test data by classifying each of a plurality of post-pace waveforms of the cardiac electrical signal episode according to a capture type selected as being one or more of: selective conduction system capture, non-selective conduction system capture, ventricular myocardial only capture without capture of the conduction system, left bundle branch capture, partial left bundle branch capture, right bundle branch capture, partial bundle branch capture, complete His bundle capture, partial His bundle capture or loss of capture.

15. The medical device system of claim 1 further comprising a communication circuit configured to receive the cardiac electrical signal episode transmitted from an implantable medical device, the cardiac electrical signal episode including at least one cardiac electrogram signal.

16. A method comprising:
receiving a cardiac electrical signal episode sensed during a conduction system capture management test;
determining capture test data from the cardiac electrical signal episode;
comparing the capture test data to previous capture test data determined from a previous conduction system capture management test;
generating an output based on the comparing of the capture test data to the previous capture test data; and
detecting an alert condition corresponding to a change in the capture test data compared to the previous capture test data;
storing the output in a memory; and
displaying data in a user interface of a display unit based on the generated output and the alert condition.

17. The method of claim 16 further comprising:
determining the capture test data by identifying a number of different QRS waveform morphologies in the cardiac electrical signal episode; and
detecting the alert condition by determining that the number of different QRS waveform morphologies in the cardiac electrical signal episode is different than a previous number of QRS waveform morphologies identified in a previous cardiac electrical signal episode sensed during the previous conduction system capture management test.

18. The method of claim 17 wherein determining the capture test data comprises determining a capture threshold for one or more of the different QRS morphologies identified in the cardiac electrical signal episode.

19. The method of claim 18 wherein detecting the alert condition comprises determining that a capture threshold determined for at least one of the QRS morphologies identified in the cardiac electrical signal episode is different than a previously determined capture threshold determined for the respective QRS morphology identified in the previous cardiac electrical signal episode sensed during the previous capture management test.

20. The method of claim 16 wherein determining the capture test data comprises classifying at least one QRS waveform in the cardiac electrical signal episode according to a capture type.

21. The method of claim 20 further comprising:
determining the capture test data by classifying a plurality of QRS waveforms in the cardiac electrical signal episode according to a plurality of capture types;
determining a capture threshold of one or more of the classified capture types of the plurality of capture types of the cardiac electrical signal episode; and
detecting the alert condition by determining at least one of:
a change in the capture threshold determined for at least one of the classified capture types compared to a previous capture threshold determined for the at least one of the classified capture types in the previous conduction system capture management test; or
determining that a classified capture type of the plurality of capture types occurs at a pacing pulse output of a plurality of pacing pulse outputs of the conduction system capture management test and that the classified capture type of the plurality of capture types does not occur at the pacing pulse output of the plurality of pacing pulse outputs of the previous conduction system capture management test.

22. The method of claim 16 further comprising receiving a user input labeling at least one QRS waveform of the cardiac electrical signal episode according to at least one of a morphology type or a capture type.

23. The method of claim 22 further comprising adjusting the determination of capture test data from the cardiac electrical signal episode in response to receiving the user input.
